(19) United States
(12) Patent Application Publication
(10) Pub. No.: US 2009/0128010 A1
(43) Pub. Date: May 21, 2009

(54) Green Electroluminescent Compounds and Organic Electroluminescent Device Using the Same

(76) Inventors: Seung-Hak Hyun, Chungnam (KR); Jea-Sung Lee, Kyeonggi-do (KR); Sang-Man Si, Seoul (KR); Keun-Hee Han, Kyeonggi-do (KR); Hyuck-Joo Kwon, Seoul (KR); Young-Jun Cho, Seoul (KR); Seung-Soo Yoon, Seoul (KR); Bong-Ok Kim, Seoul (KR); Sung-Min Kim, Seoul (KR); Chi-Sik Kim, Seoul (KR); Il-Won Choi, Seoul (KR)

Correspondence Address: CLARK & BRODY, 1090 VERMONT AVENUE, NW, SUITE 250, WASHINGTON, DC, 20005, US

(21) Appl. No.: 11/990,502

(22) PCT Filed: Aug. 14, 2006
(86) PCT No.: PCT/KR2006/003188

§ 371 Date: Feb. 15, 2008

(30) Foreign Application Priority Data
Aug. 16, 2005 (KR) 10-2005-0074983
Aug. 8, 2006 (KR) 10-2006-0074910

(51) Int. Cl. C07C 211/61; H01J 1/63

(52) U.S. Cl. 313/504; 564/427

(57) ABSTRACT

The present invention relates to organic electroluminescent compounds represented by Chemical Formula 1 or 2, a process for preparing the same, and an organic light emitting diode (OLED) which comprises, as a luminescent region interposed between an anode and a cathode, at least one compound (s) selected from those represented by Chemical Formula 1 or 2, and at least one compound selected from anthracene derivatives, benz[a]anthracene derivatives and naphthacene derivatives. The electroluminescent compound according to the present invention is a green electroluminescent compound having maximized electroluminescent efficiency and lifetime of device.

### FIELD OF THE INVENTION

The present invention relates to organic electroluminescent compounds represented by Chemical Formula 1 or 2, a process for preparing the same, and an organic light emitting diode (OLED) which comprises, as a luminescent region interposed between an anode and a cathode, at least one compound(s) selected from those represented by Chemical Formula 1 or 2, and at least one compound selected from anthracene derivatives, benz[a]anthracene derivatives and naphthacene derivatives.

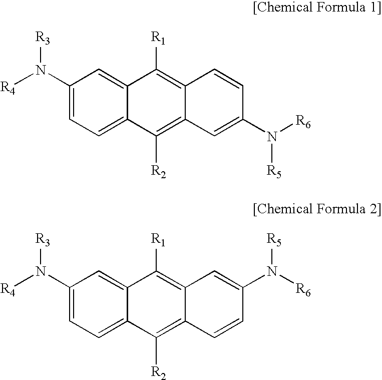

### BACKGROUND OF THE RELATED ART

The most important matter in developing an OLED having high efficiency and long life is development of electroluminescent material of high performance. In view of current development of electroluminescent material, green electroluminescent materials show superior electroluminescent property to red or blue electroluminescent materials. However, conventional green electroluminescent materials still have many problems to achieve manufacturing panels of large scale with low power consumption. In view of practical efficiency and life, various kinds of materials for green have been reported up to now. Though they show from 2 to 5 times of electroluminescent property as compared to red or blue electroluminescent materials, development of green electroluminescent material become a burden with the improvement of properties of red or blue electroluminescent material. In the meanwhile, enhancement of lifespan of the green material is still insufficient, so that a green electroluminescent material having long life is seriously required.

As green fluorescent material, a coumarin derivative (Compound D), a quinacridone derivative (Compound E), DPT (Compound F) and the like have been known. Compound D is the structure of C545T that is the most widely used coumarin derivative up to the present. In general, those materials are doped at from several % to not more than 20% concentration by using Alq as the host to form an EL diode.

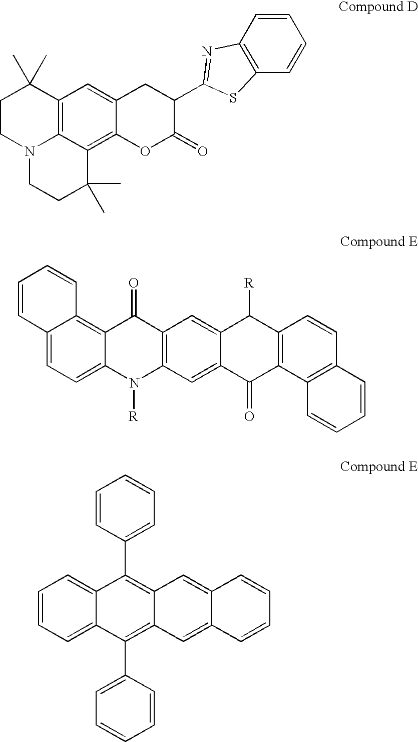

In Japanese Patent Laid-Open No. 2001-131541, disclosed are bis(2,6-diarylamino)-9,10-diphenylanthracene derivatives represented by following Chemical Formula G, in which diarylamino groups are directly substituted at 2- and 6-position of anthracene, respectively.

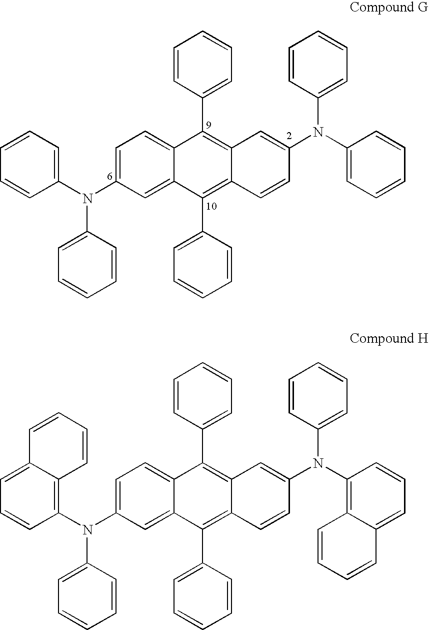

Japanese Patent Laid-Open No. 2003-146951 disclosing the compounds for hole transport does not describe those compounds wherein diarylamino groups are directly substituted at 2- and 6-position, but having phenyl groups at 9- and 10-position of anthracene; and Japanese Patent Laid-Open No. 2003-146951 recognizes that Compound H having diarylamino groups directly substituted at 2- and 6-position of anthracene ring shows poor electroluminescent efficiency. In view of these facts, the invention of Japanese Patent Laid-Open No. 2003-146951 does not recognize the compounds other than those having phenyl substituents at 9- and 10-position of anthracene. In order to overcome the problems described above, Japanese Patent Laid-Open No. 2003-146951 has suggested an electroluminescent compound represented by following Chemical Formula 1 having about twice of the electroluminescent efficiency, on the basis of recognizing the fact that the electroluminescent efficiency is enhanced if one diarylamino group is substituted at 2-position of anthracene and an aryliminophenyl group is substituted at 6-position.

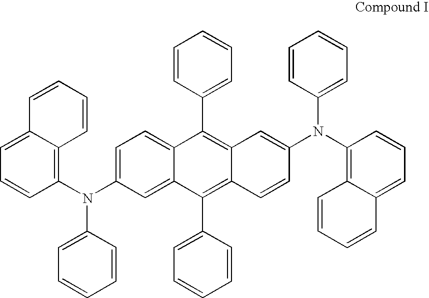

However, the above-suggested compound has problems of low hole transport property and insufficient luminance, though the electroluminescent efficiency has been increased. Those materials are not employed as electroluminescent material with limitation to be applied as practical electroluminescent material, because Compound I emits light of light-blue color with low electroluminescent efficiency.

Japanese Patent Laid-Open No. 2004-91334 suggested an organic electroluminescent compound represented by following Chemical Formula J having low ionization potential and excellent hole transport property with overcoming low electroluminescent efficiency of the conventional compounds, by further substituting the aryl group of the diarylamino group with another diarylamino group, though said diarylamino group being already directly substituted to anthracene.

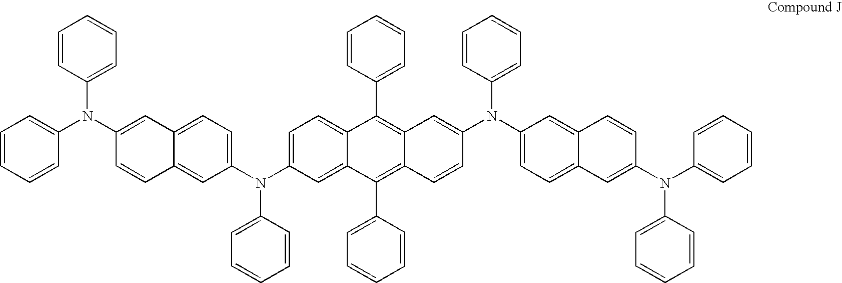

Though the compounds suggested by Japanese Patent Laid-Open No. 2004-91334, applied as a hole transport layer, could lower the ionization potential and enhance the hole transport property due to many amine functional groups, they have problem of short lifetime of operation as a hole transport layer because of multiple amine functional groups. In the detailed description of the invention of Japanese Patent Laid-Open No. 2004-91334, disclosed are some compounds having 1-naphthyl and 9-phenanthryl group at 9- and 10-position of anthracene: but the facts that blue shift phenomenon is involved in a structure having a multi-cycle of α-type at 9- and 10-position of anthracene to result in lowered electroluminescent efficiency, and that the inventors could not recognize the electroluminescent efficiency in case that a fused multi-cyclic aromatic ring is actually substituted at 9- and 10-position of anthracene reflect that they did not specifically practiced those compounds.

In the meanwhile, U.S. Pat. No. 6,465,115 discloses an organic multi-layer electroluminescent device characterized by a hole transport layer comprising the following organic compounds between the anode and cathode.

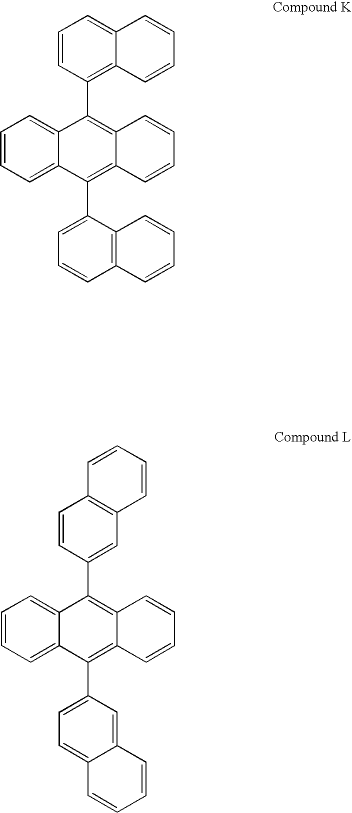

However, U.S. Pat. No. 6,465,115 did not employed Compound K and Compound L in the luminescent region, and could not confirm the properties of those materials in the luminescent region. In particular, they could not recognize that the derivatives having the substituent according to the present invention at 2-position provide far better electric property than the derivatives simply having aromatic substituents at 9- and 10-position of anthracene do.

The present inventors now confirmed that the derivatives having a substituent at 2-position of 9,10-diarylanthracene extraordinarily enhance the electroluminescent property of compounds of Chemical Formula 1 or 2, and completed the present invention.

Surprisingly, the present inventors found that simple introduction of a multi-cyclic aromatic ring such as naphthalene to 9- and 10-position of anthracene overcomes the problems of the conventional hole transport materials including lowering of electroluminescent efficiency, shortening of operation life of device and increasing of ionization potential, even though diarylamino groups are directly substituted at 2- and 6-position of the anthracene ring; and they incorporated the construction that applies those compounds as electroluminescent material, to complete the invention. This eventually was not able to be recognized in any prior invention including Japanese Patent Laid-Open No. 2003-146951 and 2004-91334. In addition, the present inventors found that when at least one compound selected from anthracene derivatives, benz[a]anthracene derivatives and naphthacene derivatives is(are) employed as a light-emitting host in luminescent region together with at least one compound of Chemical Formula 1, enhancement of color reproducibility due to improvement of color purity and noticeable increase of electroluminescent efficiency, as well as increased lifetime of device.

### DISCLOSURE

### Technical Problem

The object of the present invention is to provide a novel organic electroluminescent compound in which a fused multi-cyclic aromatic ring such as naphthalene, anthracene or fluoranthene is substituted at 9- and 10-position of anthracene, and diarylamino groups are directly substituted at 2- and 6-position of anthracene ring, respectively. Another object of the present invention is to provide an organic light emitting diode which has a luminescent region employing at least one compound selected from anthracene derivatives, benz[a]anthracene derivatives and naphthacene derivatives together with at least one compound of Chemical Formula 1, as the light emitting host. Still another object of the invention is to provide an organic electroluminescent compound having excellent color purity, good electroluminescent efficiency and long life of the device, and to provide an OLED containing the novel organic electroluminescent compound as described above.

### Technical Solution

The present invention relates to organic electroluminescent compounds represented by Chemical Formula 1 or 2, and a process for preparing the same.

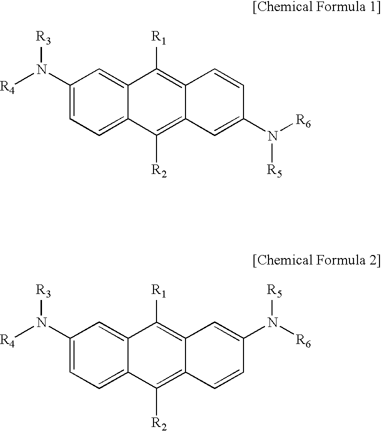

In the Chemical Formula 1 and 2, R₁ and R₂ independently represent a fused multi-cyclic aromatic ring having two or more aromatic rings fused therein, and R₃ to R₆ independently represent an aromatic ring, and each aromatic ring of R₁ to R₆ may be further substituted by a C1-C20 alkyl group, a C1-C20 alkoxy group, a halogen atom or a C5-C7 cycloalkyl group.

In addition, the present invention relates to an organic light emitting diode (OLED) comprising a first electrode, at least one organic layer and a second electrode in a subsequently laminated form, wherein at least one layer of said organic layers comprise(s) the organic electroluminescent compound of Chemical Formula 1 or 2. Further, the present invention relates to an OLED comprising an anode; a cathode; and a luminescent region interposed between said anode and said cathode, wherein said luminescent region comprises at least one organic electroluminescent compound represented by Chemical Formula 1 or 2; and at least one compound selected from anthracene derivatives, benz[a]anthracene derivatives and naphthacene derivatives.

Other and further objects, features and advantages of the invention will appear more fully from the following description.

The compounds of Chemical Formula 1 or 2 according to the present invention are characterized by the structure of novel concept with maximized electroluminescent efficiency in green light-emitting diode and lifetime of device which were not expectable with conventional inventions.

The compounds of Chemical Formula 1 or 2 according to the invention adopted a structure showing an efficient energy transfer mechanism between the host and the dopant, which can reveal electroluminescent property with a reliably high efficiency on the basis of improvement in electron density distribution. The structure of the novel compounds according to the present invention can provide a skeletal which can also tune an electroluminescent property with high efficiency in the range from blue to red, not only for green light. Beyond the concept of using a host material with high electric conductivity such as Alq, the invention applies a host having appropriate balance of hole conductivity and electron conductivity, thereby overcoming the problems of conventional materials including low initial efficiency and short lifetime, and ensures the electroluminescent property with high performance having high efficiency and long life for each color.

As can be seen from FIG. 1 and FIG. 2 showing the electron density distribution of the compound according to the invention wherein amine groups are incorporated to 2- and 6-position of anthracene and 2-naphthyl group as a fused multi-cyclic aromatic group are substituted at 9- and 10-position, and the electron density distribution of the compound wherein aromatic rings are incorporated to 2- and 6-position of anthracene, respectively, when an amine group is substituted at i-position (2- and 6- or 7-position) of anthracene, electroluminescent property with high efficiency is obtained due to even electron distribution up to the side branch of core skeletal; while when an aromatic ring is directly positioned on the core skeletal, the electron density of the side branch noticeably falls down. This illustrates that amine groups should be directly incorporated to the core skeletal in order to get electroluminescent property with high efficiency.

These results show that the electroluminescent material according to the prior art, in which an aromatic ring is employed as a spacer only for the purpose of tuning the light-emitting wavelength, inevitably has the limitation in improving the electroluminescent efficiency.

According to the present invention, electroluminescent materials having at least twice of efficiency of that of conventional material are developed by using a method of directly incorporating amine group at β-position and a concept of incorporating multi-cyclic aromatic ring at 9- and 10-position of core anthracene in order to overcome the problems mentioned above.

As described heretofore, in case of Compound G and Compound H exemplified in Japanese Patent Laid-Open No. 2003-146951 having similar structure to that of Chemical Formula 1 according to the present invention, wherein diarylamino groups are directly substituted at 2- and 6-position, respectively, and phenyl groups are substituted at 9- and 10-position, the problem is low electroluminescent efficiency. According to the present inventors, the problem is caused by disadvantageous structure for energy transfer with the host, and said compounds suggested by prior inventions inevitably have limitation that they cannot improve the properties of dopant at all no matter how good the property of the host is.

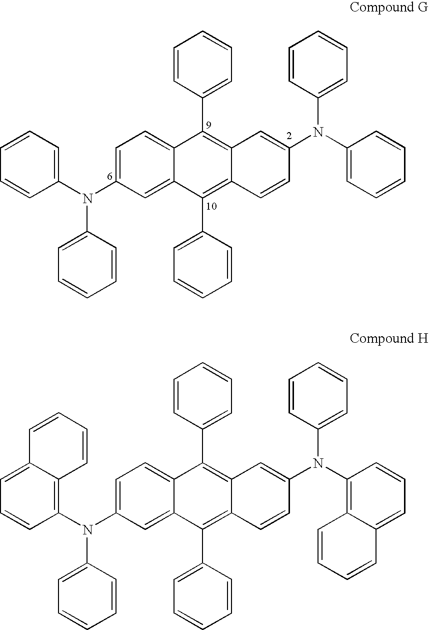

On the basis of research results as described above, the inventors found that, even though the longer wavelength shift property due to simple overlap of molecules cannot be overcome with the size and 3-dimensional structural property like phenyl, when diarylamino groups are directly substituted at 2- and 6-position of anthracene, respectively, and phenyl groups at 9- and 10-position, the overlap of n-electron with other molecules are very efficiently achieved by incorporating a fused multi-cyclic aromatic ring (at least naphthalene) at 9- and 10-position of anthracene in the compounds of Chemical Formula 1 or 2 according to the present invention, thereby much enhancing the energy transfer properties. The present invention is established on the basis of such discoveries.

Thus, the compounds represented by Chemical Formula 1 or 2 according to the present invention are characterized in that a diarylamine group having an aromatic ring at the β-position of anthracene is directly substituted, and a fused multi-cyclic aromatic ring with two or more aromatic ring fused is substituted for R₁ and R₂ at 9- and 10-position. Preferably, the fused multi-cyclic aromatic ring independently represents naphthyl, anthryl, fluoranthenyl, pyrenyl, fluorenyl, biphenyl and perilenyl group. Groups R₃ to R₆ that are substituted for amine substituted at β-position of anthracene independently represent phenyl, naphthyl, anthryl, phenanthryl, fluorenyl, fluoranthenyl, pyrenyl, perilenyl, naphthacenyl or biphenyl group.

More preferably, the fused multi-cyclic aromatic ring of R₁ and R₂ in Chemical Formula 1 or 2 is independently selected from 2-naphthyl, 2-anthryl, 2-fluoranthenyl, 1-pyrenyl, 2-fluorenyl, 4-biphenyl and 3-perilenyl group. Due to the substitution of said fused multi-cyclic aromatic ring at a certain position, optimal overlap of n-electrons of the fused multi-cyclic aromatic ring with other molecules can be achieved, and the selection of the position for the fused multi-cyclic aromatic ring compound also is an important feature of the invention.

In order to improve electroluminescent properties of the compounds according to the present invention, the aromatic rings of R₃ to R₆ independently may have a substituent selected from a C1-C20 alkyl group, a C1-C20 alkoxy group, a halogen atom and C5-C7 cycloalkyl group. Preferably, each aromatic group of R₁ to R₆ preferably is substituted by methyl, t-butyl or methoxy group.

Among the compounds represented by Chemical Formula 1 or 2 according to the present invention, compounds having one of the following chemical structures are preferably mentioned.

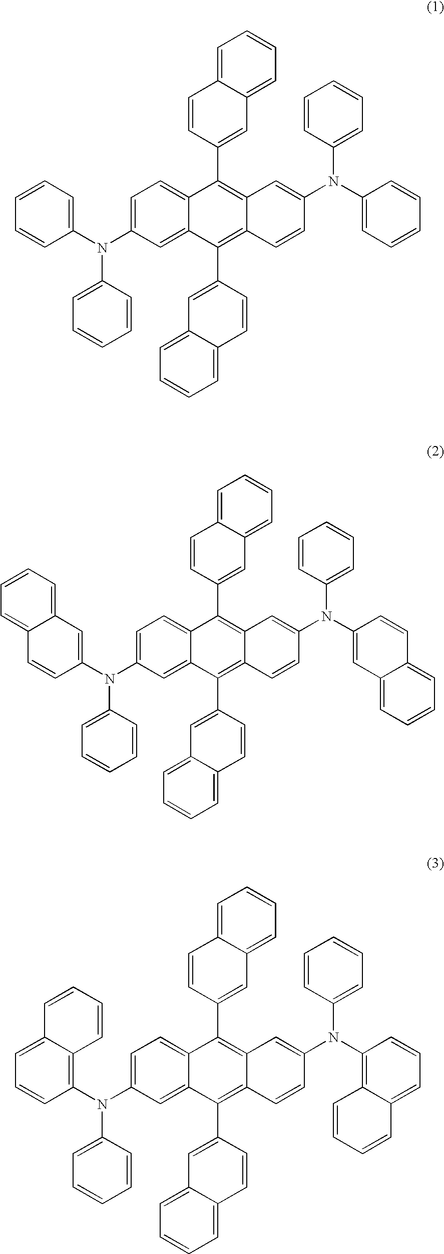

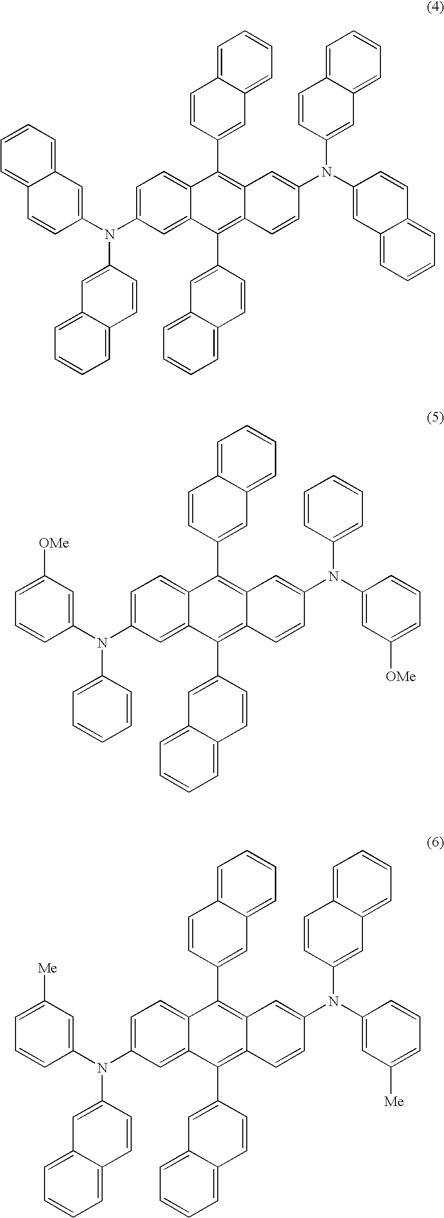

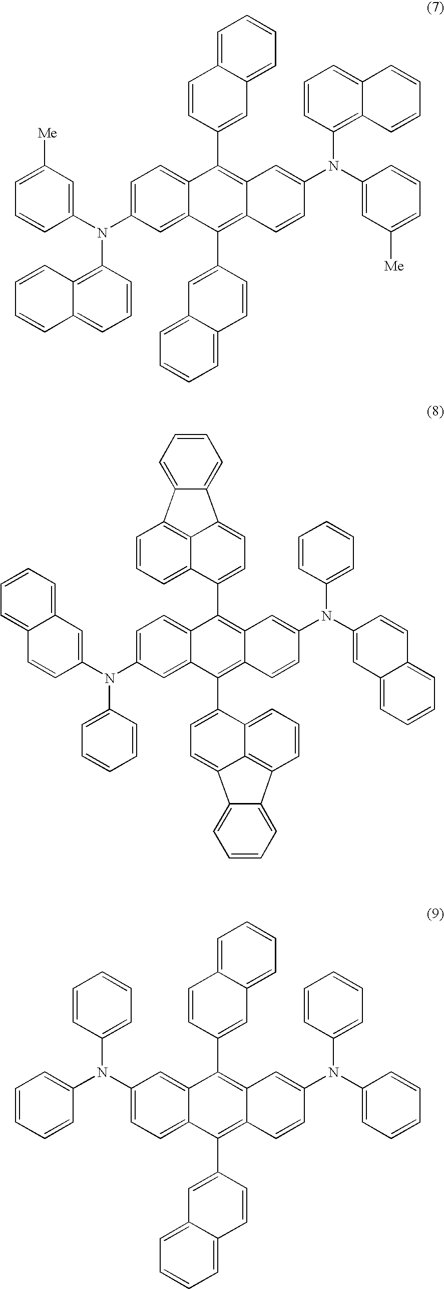

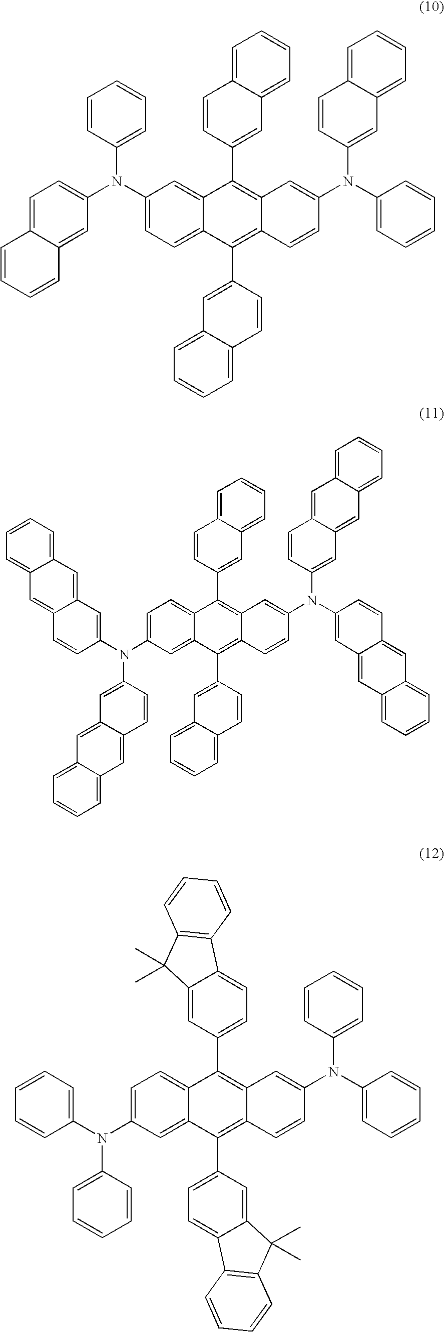

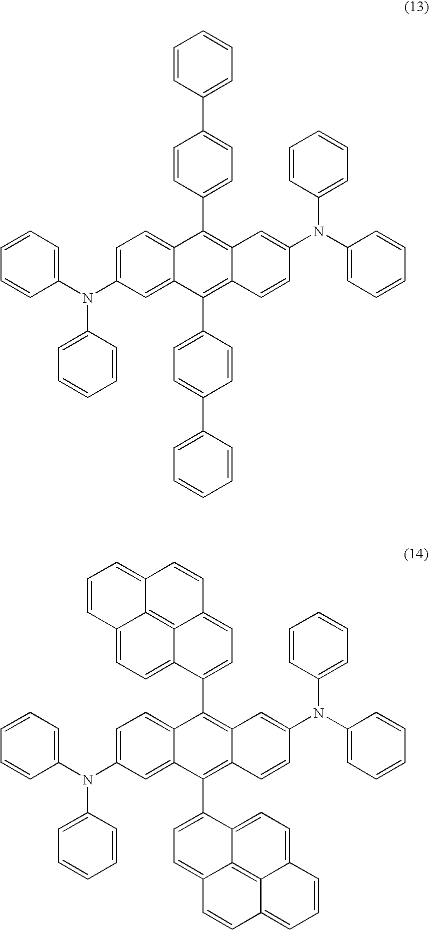

The compound represented by Chemical Formula 1 or 2 according to the present invention can be prepared as shown in Reaction Scheme 1: 2,6-dihaloanthraquinone (2,6-DHAQ) or 2,7-dihaloanthraquinone is reacted with diarylamine to obtain bis(diarylamino)anthraquinone (BDAAQ); BDAAQ is then treated with lithium compound of fused multi-cyclic aromatic compound to give dihydroanthracenediol compound (DHAD); DHAD is dehydrated to completely form the anthracene skeletal.

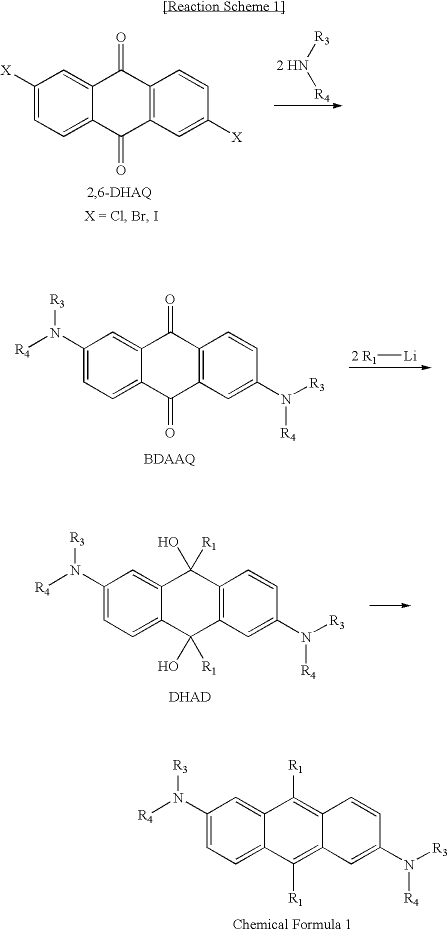

In addition, the present invention relates to an organic light emitting diode (OLED) comprising a first electrode, at least one organic layer and a second electrode in a subsequently laminated form, wherein at least one layer of said organic layers comprise(s) the organic electroluminescent compound of Chemical Formula 1 or 2. Further, the present invention relates to an organic light emitting diode comprising an anode; a cathode; and a luminescent region interposed between said anode and said cathode, wherein said luminescent region comprises at least one organic electroluminescent compound of Chemical Formula 1 or 2; and at least one compound selected from anthracene derivatives, benz[a]anthracene derivatives and naphthacene derivatives.

The luminescent region means the layer where light emitting occurs. The layer may be single layer or multiple layers with two or more layers laminated. According to the construction of the invention, when the host-dopant is employed together with the compound of Chemical Formula 1 or 2, noticeable improvement of electroluminescent efficiency could be confirmed due to the luminescent host, on the contrary of the case using the compound of Chemical Formula 1 or 2 only. The host-dopant, which can be constructed with a doping concentration of 2 to 5%, has very excellent conductivity with regard to the holes and electrons as compared to other conventional host materials, and very excellent material stability to result in improving the lifetime as well as electroluminescent efficiency.

Thus it is understand that if a compound selected from anthracene derivatives, benz[a]anthracene derivatives and naphthacene derivatives is employed as a luminescent host, electric disadvantage of the compound of Chemical Formula 1 or 2 of the invention may be significantly complemented.

The anthracene derivatives or benz[a]anthracene derivatives contained with at least one organic electroluminescent compound of Chemical Formula 1 or 2 in said luminescent region include the compounds represented by Chemical Formula 3 or 4:

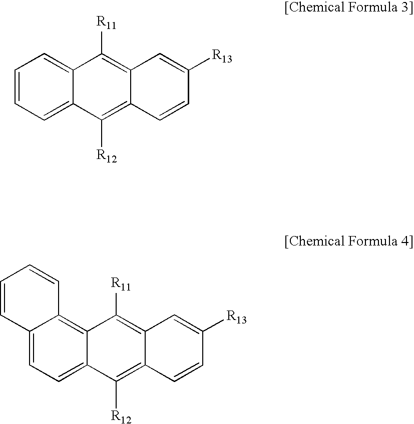

wherein, each of R₁₁ and R₁₂ independently represents a C6-C20 aromatic ring or a fused multi-cyclic aromatic ring, R₁₃ represents a hydrogen, a C1-C20 alkyl group, a C1-C20 alkoxy group, a halogen atom, a C5-C7 cycloalkyl group, or a C6-C20 aromatic ring or a fused multi-cyclic aromatic ring, and each aromatic ring of R₁₁ to R₁₃ may have further substituent(s) of a C1-C20 alkyl group, a C1-C20 alkoxy group, a halogen atom or a C5-C7 cycloalkyl group.

The compounds represented by Chemical Formula 3 or 4 may be exemplified by the compounds in which R₁₁ to R₁₃ independently represent phenyl, 2-naphthyl, 2-anthryl, 2-fluoranthenyl, 1-pyrenyl, 2-fluorenyl, 4-biphenyl and 3-perilenyl group.

The anthracene derivatives include compounds of following formulas:

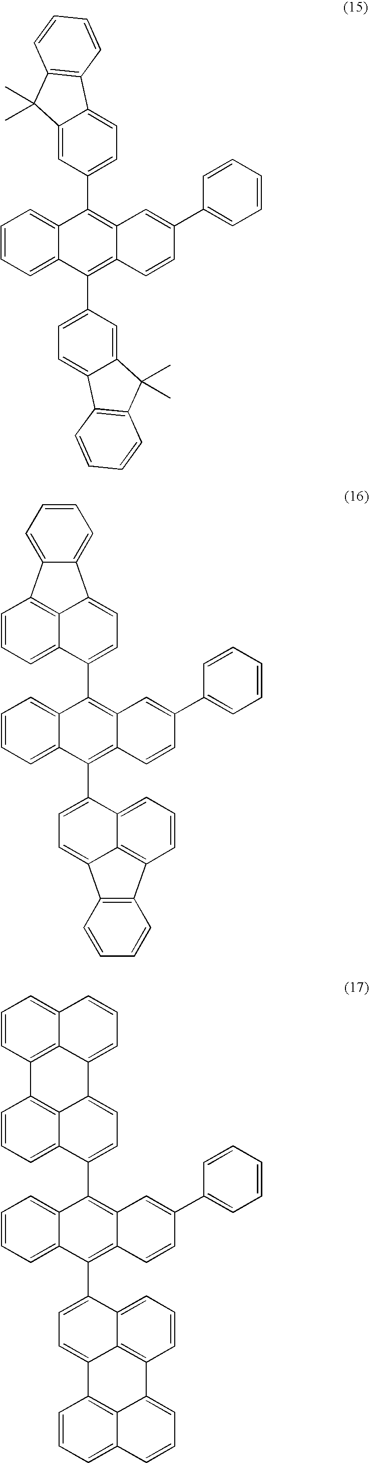

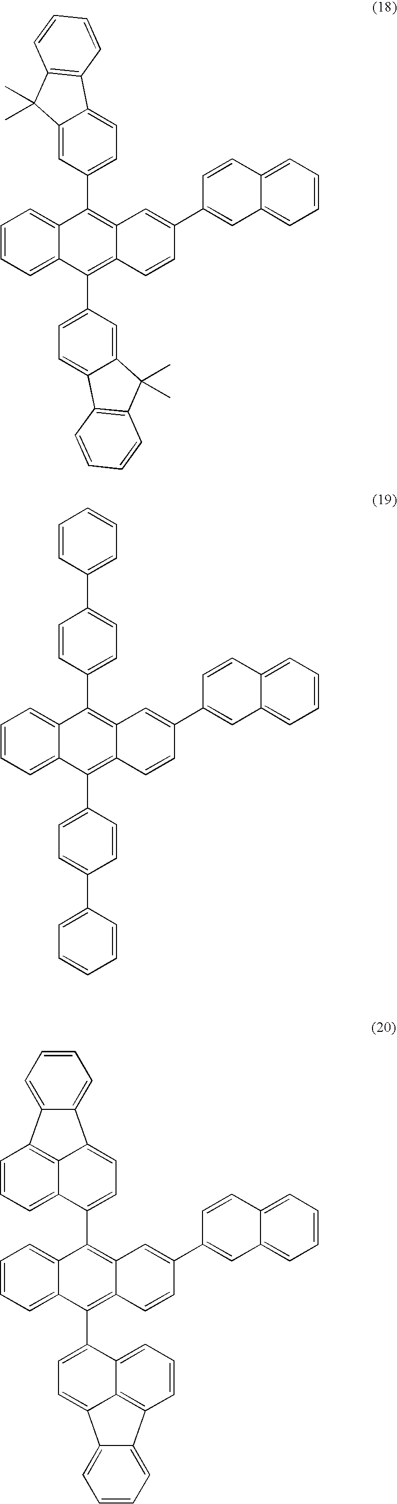

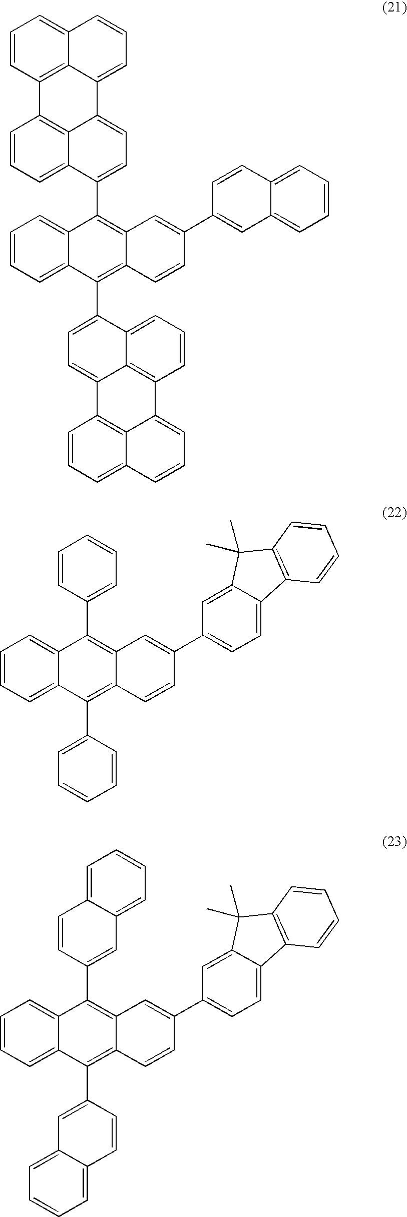

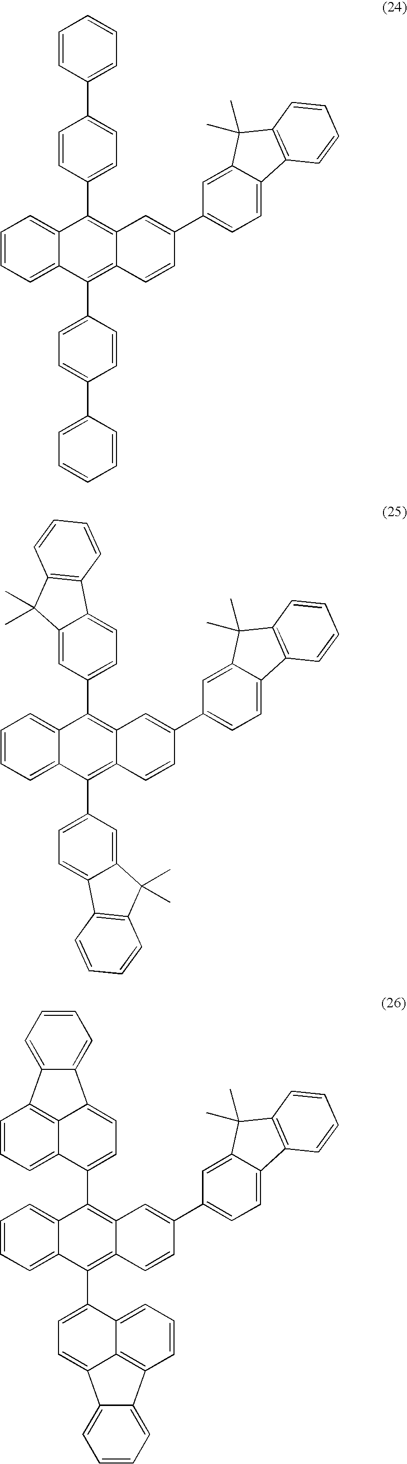

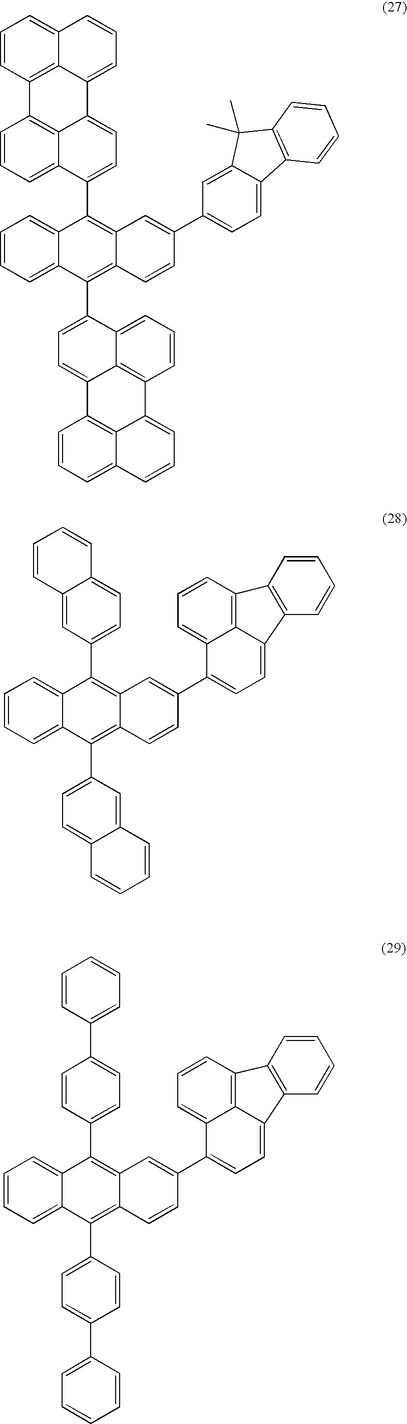

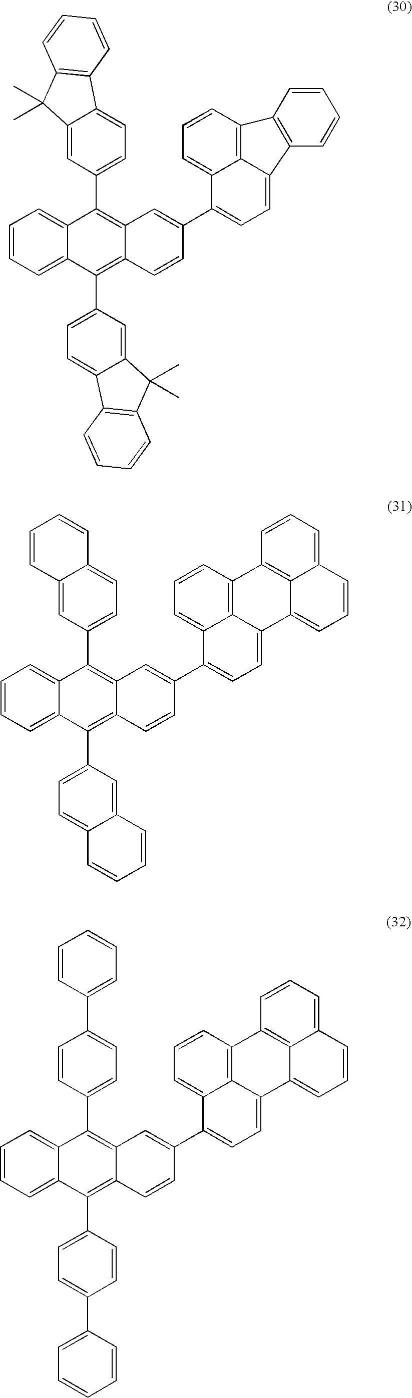

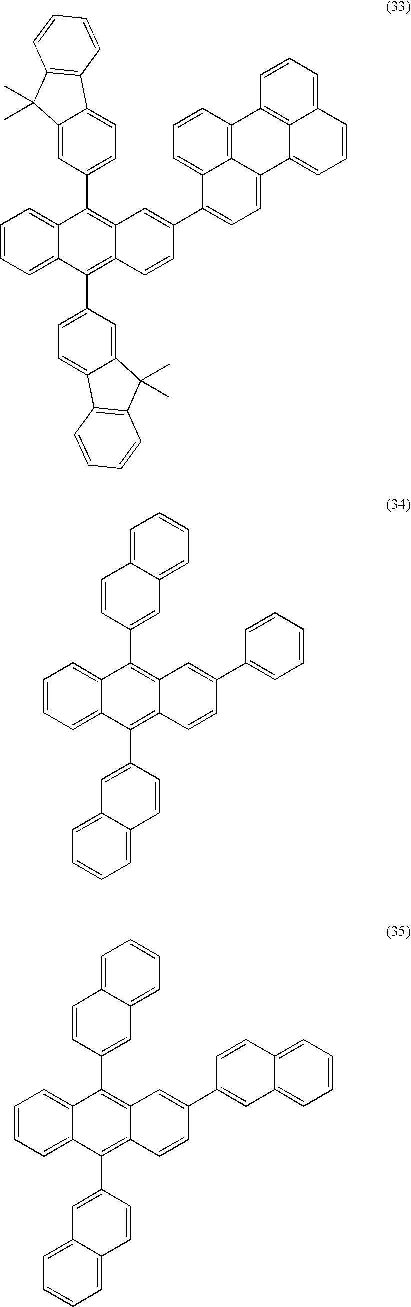

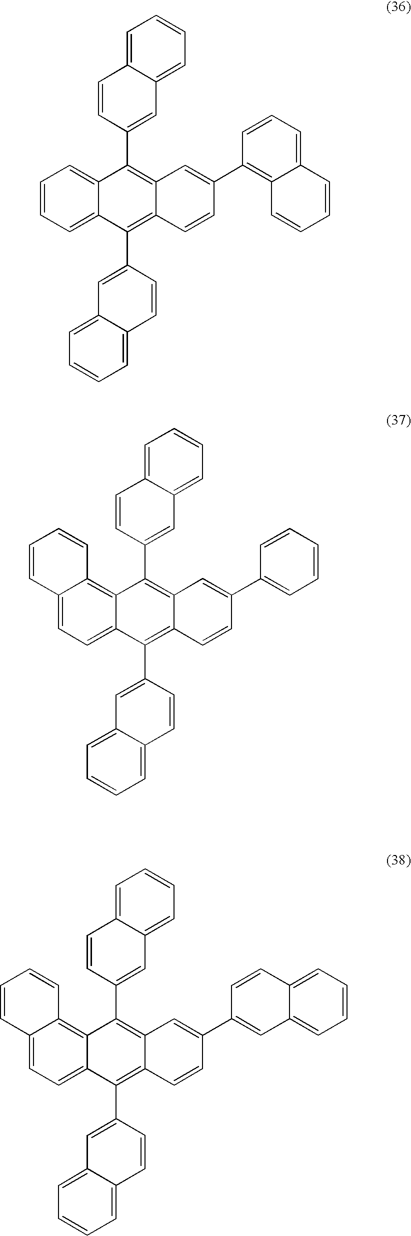

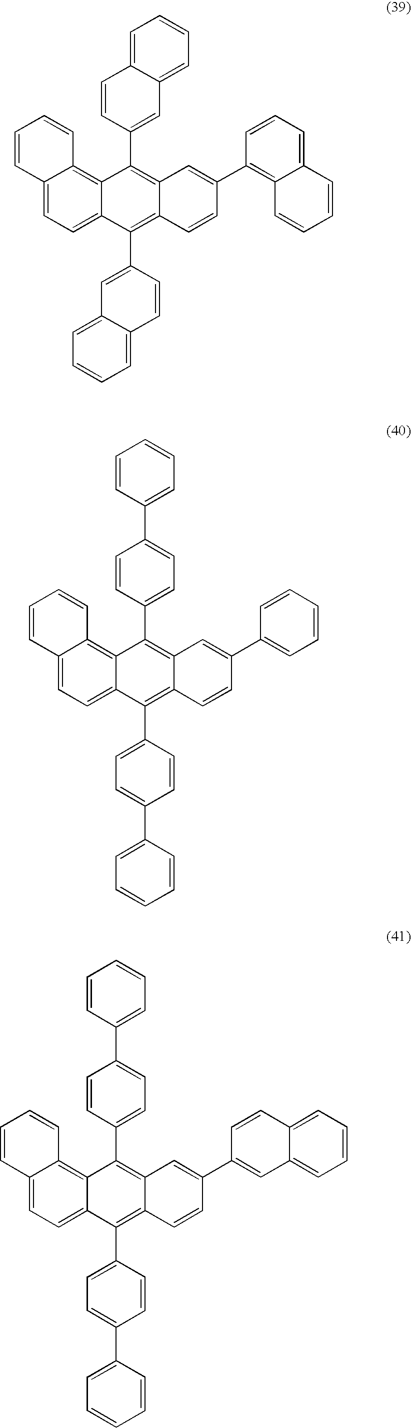

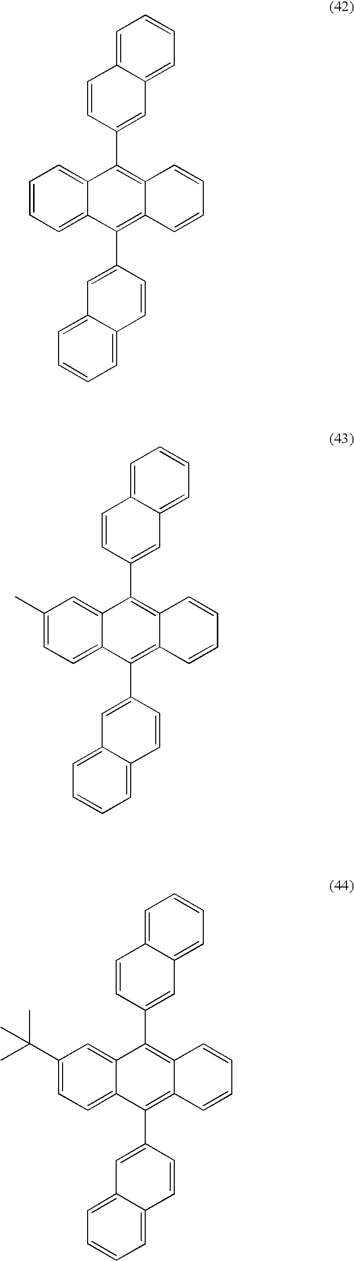

### BRIEF DESCRIPTION OF DRAWINGS

FIG. 1 illustrates distribution of electron density of the compound according to the present invention.

FIG. 2 illustrates distribution of electron density of the compound wherein aromatic rings are incorporated at 2- and 6-position of anthracene.

FIG. 3 shows the change of electroluminescent efficiency versus luminance of an OLED employing Alq and C545T as the luminescent materials.

FIG. 4 shows the change of electroluminescent efficiency versus luminance of an OLED from Comparative Example 2.

FIG. 5 shows the change of luminance versus operation voltage of an OLED employing Compound 4 according to the present invention and DNPBA as the electroluminescent materials.

FIG. 6 shows the change of electroluminescent efficiency versus luminance of OLED employing Compound 4 according to the present invention and DNPBA as the electroluminescent materials.

FIG. 7 is an EL spectrum of an OLED employing Compound 4 according to the present invention and DNPBA as the electroluminescent materials.

FIG. 8 shows the change of CIE coordinate versus luminance between the OLED employing Compound 4 according to the present invention and DNPBA as the electroluminescent materials and the OLED from Comparative Example 1 or 2.

FIG. 9 shows the life curves of the OLED from Example 1 according to the present invention and the OLED from Comparative Example 1 or 2.

FIG. 10 shows the change of electroluminescent efficiency versus luminance of the OLEDs employing Compound 23 and Compound 1 of the present invention.

FIG. 11 shows the change of CIE coordinate versus luminance of the OLEDs employing Compound 23 and Compound 1 of the present invention as the electroluminescent material.

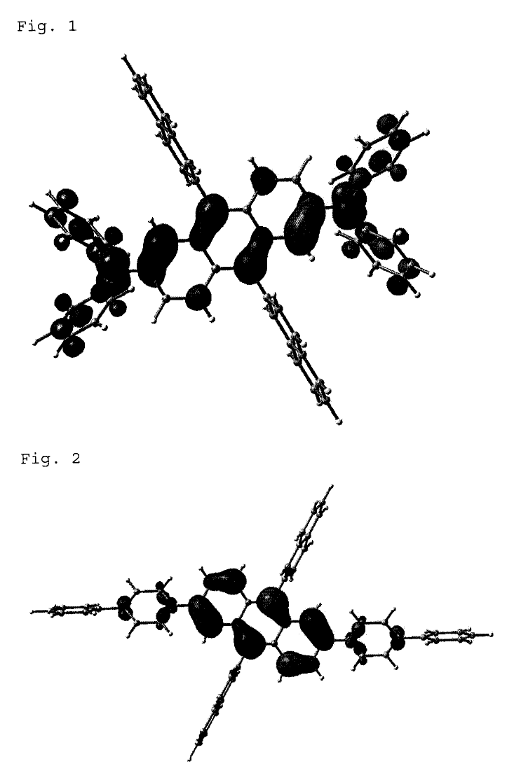

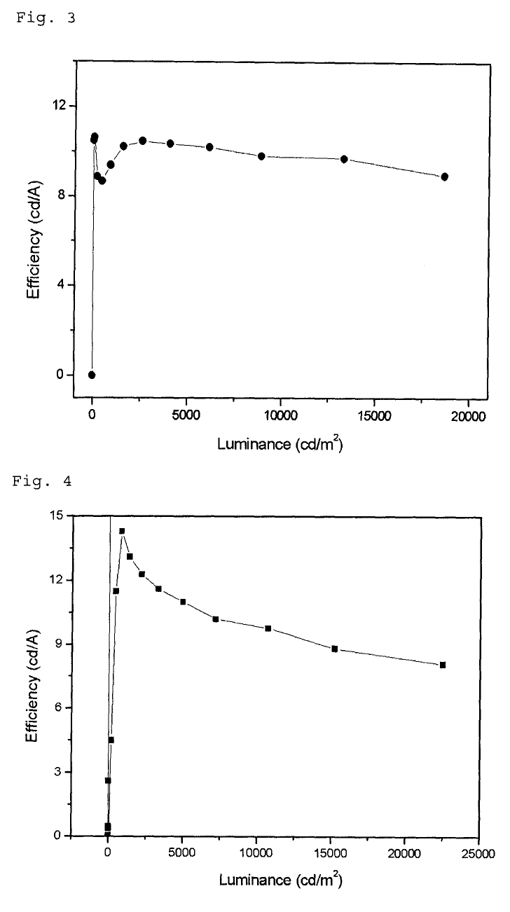

### MODE FOR INVENTION

### Examples

The present invention is further described with respect to the compounds according to the invention, a process for preparing the same and the electroluminescent properties of the device employing the same by referring to representative compounds according to the present invention, which are provided for illustration only and are not intended to be limiting in any way.

### Preparation Example 1

### Preparation of Compound (1) (Chemical Formula 1: R₁=R₂=2-naphthayl, R₃=R₄=R₅=R₆=phenyl)

In dry toluene, dissolved were 2,6-dichloroanthraquinone (1.0 g, 3.6 mmol) and diphenylamine (1.3 g, 7.7 mmol), and palladium acetate (Pd(OAc)₂)(2.4 g, 24.4 mmol), tri(t-butyl) phosphine (P(t-Bu)₃) (0.2 mL, 1.9 mmol) and sodium t-butoxide (t-BuONa) (0.93 g, 9.7 mmol) were added thereto. The resultant mixture was heated under reflux at 110° C. for 3 days. When the reaction was completed, 10 mL of distilled water was added, and the mixture was stirred for 30 minutes. The solid generated was filtered, washed with solvent such as acetone and THF, dried and recrystallized from methylene chloride to give bis(2,6-diphenylamino)anthraquinone (1.1 g, 2.0 mmol, yield: 56%).

Diethyl ether solution (5 mL) of 2-naphthyllithium which had been previously prepared by using diphenylamine (0.74 g, 4.4 mmol) and n-buthyllithium (n-BuLi) (1.8 mL, 4.5 mmol, 2.5 M in hexane) was slowly added to a solution of bis(2,6-diphenylamino)anthraquinone obtained as described above (1.1 g, 2.0 mmol) in dry THF (30 mL) at −78° C. under nitrogen atmosphere. The reaction mixture was stirred for 2 hours at the same temperature, and warmed to ambient temperature, before stirring for 12 hours. Saturated aqueous ammonium chloride solution (30 mL) was added thereto, and the resultant mixture was stirred for 2 hours to complete the reaction. The resultant solid was filtered, washed with acetone and dried to give 2,6-bis(diphenylamino)-9,10-[di-(2-naphthyl)]-9,10-dihydro-9,10-anthracenediol (1.3 g, 1.7 mmol, yield: 85%).

The diol compound (1.3 g, 1.71 mmol) thus obtained was added to 30 mL of acetone, and potassium iodide (1.6 g, 7.8 mmol) and sodium hydrogen phosphate monohydrate (2.0 g, 14.5 mmol) were added thereto. The resultant mixture was heated under reflux for 12 hours. When the reaction was completed, an equivalent volume of distilled water was added thereto to form precipitate, which was then filtered, washed with water and acetone to give solid product. After recrystallization from THF, the title compound (1) (0.68 g, 0.89 mmol, yield: 52%) was obtained.

¹H NMR (200 MHz, CDCl₃): δ 6.46 (d, 8H), 6.65-6.75 (m, 8H), 7.0 (m, 8H), 7.3 (m, 4H), 7.5-7.6 (m, 4H), 7.65-7.8 (m, 6H), 7.9 (s, 2H)

MS/FAB: 764 (found), 764.98 (calculated)

### Preparation Example 2

### Preparation of Compound (2) (Chemical Formula 1: R₁=R₂=R₃=R₅=2-naphthayl, R₄=R₆=phenyl)

The same procedure as described in Preparation Example 1 was repeated but using N-phenyl-2-naphthylamine (1.7 g, 7.8 mmol) to obtain Compound (2) (0.53 g, 0.61 mmol, overall yield: 17%).

¹H NMR (200 MHz, CDCl₃): δ 6.45 (d, 4H), 6.6 (t, 2H), 6.75-6.8 (m, 8H), 7.0-7.15 (m, 6H), 7.2-7.3 (m, 6H), 7.45-7.6 (m, 10H), 7.65-7.8 (m, 6H), 7.9 (s, 2H)

MS/FAB: 864 (found), 865.10 (calculated)

### Preparation Example 3

### Preparation of Compound (3) (Chemical Formula 1: R₁=R₂=2-naphthyl, R₃=R₅=1-naphthyl, R₄=R₆=phenyl)

The same procedure as described in Preparation Example 1 was repeated but using N-phenyl-1-naphthylamine (1.7 g, 7.8 mmol) to obtain Compound (3) (0.41 g, 0.47 mmol, overall yield: 13%).

¹H NMR (200 MHz, CDCl₃): δ 6.45 (d, 4H), 6.5 (d, 2H), 6.6 (t, 2H), 6.75-6.8 (m, 4H), 7.0-7.05 (m, 4H), 7.15-7.2 (m, 4H), 7.3-7.35 (m, 8H), 7.55-7.8 (m, 14H), 7.9 (s, 2H)

MS/FAB: 864 (found), 865.10 (calculated)

### Preparation Example 4

### Preparation of Compound (4) (Chemical Formula 1: R₁=R₂=R₃=R₄=R₅=R₆=2-naphthyl)

The same procedure as described in Preparation Example I was repeated but using di(2-naphthyl)amine (2.1 g, 7.8 mmol) to obtain Compound (4) (0.52 g, 0.54 mmol, overall yield: 15%).

¹H NMR (200 MHz, CDCl₃): δ 6.75-6.8 (m, 12H), 7.0-7.1 (m, 4H), 7.2-7.35 (m, 8H), 7.45-7.6 (m, 16H), 7.65-7.8 (m, 6H), 7.9 (s, 2H)

MS/FAB: 964 (found), 965.22 (calculated)

### Preparation Example 5

### Preparation of Compound (5) (Chemical Formula 1: R₁=R₂=2-naphthyl, R₃=R₅=phenyl, R₄=R₆=3-methoxyphenyl)

The same procedure as described in Preparation Example 1 was repeated but using 3-methoxyphenylamine (1.53 g, 7.7 mmol) to obtain Compound (5) (1.0 g, 1.21 mmol, overall yield: 34%).

¹H NMR (200 MHz, CDCl₃): δ 3.75 (s, 6H), 5.95-6.05 (m, 4H), 6.15 (d, 2H), 6.45 (d, 4H), 6.6 (t, 2H), 6.75-7.05 (m, 10H), 7.3 (m, 4H), 7.5-7.55 (m, 4H), 7.65-7.8 (m, 6H), 7.9 (s, 2H)

MS/FAB: 824 (found), 825.03 (calculated)

### Preparation Example 6

### Preparation of Compound (6) (Chemical Formula 1: R₁=R₂=R₃=R₅=2-naphthyl, phenyl, R₄=R₆=3-methylphenyl)

The same procedure as described in Preparation Example 1 was repeated but using N-m-tolyl-2-naphthylamine (1.8 g, 7.7 mmol) to obtain Compound (6) (0.61 g, 0.68 mmol, overall yield: 19%).

¹H NMR (200 MHZ, CDCl₃): δ 2.3 (s, 6H), 6.25-6.30 (t, 4H), 6.4 (d, 2H), 6.75-6.9 (m, 10H), 7.1 (m, 2H), 7.2-7.3 (m, 6H), 7.4-7.55 (m, 10H), 7.65-7.8 (m, 6H), 7.9 (s, 2H)

MS/FAB: 892 (found), 893.15 (calculated)

### Preparation Example 7

### Preparation of Compound (7) (Chemical Formula 1: R₁=R₂=2-naphthyl, R₃=R₅=1-naphthyl, phenyl, R₄=R₆=3-methylphenyl)

The same procedure as described in Preparation Example 1 was repeated but using N-m-tolyl-1-naphthylamine (1.8 g, 7.7 mmol) to obtain Compound (7) (0.38 g, 0.43 mmol, overall yield: 12%)

¹H NMR (200 MHz, CDCl₃): δ 2.3 (s, 6H), 6.25-6.3 (t, 4H), 6.4-6.5 (m, 4H), 6.75-6.9 (m, 6H), 7.15 (t, 4H), 7.3 (m, 8H), 7.5-7.8 (m, 14H), 7.9 (s, 2H)

MS/FAB: 892 (found), 893.15 (calculated)

### Preparation Example 8

### Preparation of Compound (8) (Chemical Formula 1: R₁=R₂=1-fluoranthenyl, R₃=R₅=phenyl, R₄=R₆=2-naphthyl)

The same procedure as described in Preparation Example 1 was repeated but using bis(2,6-diphenylamino)anthraquinone (1.16 g, 1.8 mmol) prepared from Preparation Example 2 with 1-bromofluoranthene (1.1 g, 3.9 mmol) to obtain Compound (8) (0.77 g, 0.76 mmol, overall yield: 21%).

¹H NMR (200 MHz, CDCl₃): δ 6.4 (d, 4H), 6.6 (t, 2H), 6.75-6.8 (m, 8H), 7.0-7.1 (m, 6H), 7.2-7.3 (m, 10H), 7.45-7.6 (m, 10H), 7.7-7.8 (m, 4H), 7.9-7.95 (m, 4H)

MS: 1012 (found), 1013.27 (calculated)

### Preparation Example 9

### Preparation of Compound (9) (Chemical Formula 2: R₁=R₂=2-naphthyl, R₃=R₄=R₅=R₆=phenyl)

The same procedure as described in Preparation Example 1 was repeated but using 2,7-dichloroanthraquinone (0.5 g, 1.8 mmol) and diphenylamine (0.65 g, 3.9 mmol) to obtain bis(2,7-diphenyl)anthraquinone (0.60 g, 1.1 mmol, yield: 61%). The same procedure as described in Preparation Example 1 was repeated but using bis(2,7-diphenyl)anthraquinone (0.6 g, 1.1 mmol) thus prepared, to obtain Compound (9) (0.40 g, 0.52 mmol, overall yield: 29%).

¹H NMR (200 MHz, CDCl₃): δ 6.4 (d, 8H), 6.6 (t, 4H), 6.75-6.8 (m, 4H), 7.0 (m, 8H), 7.3 (m, 4H), 7.5-7.55 (m, 4H), 7.65-7.8 (m, 6H), 7.9 (s, 2H)

MS: 764 (found), 764.98 (calculated)

### Preparation Example 10

### Preparation of Compound (10) (Chemical Formula 2: R₁=R₂=R₃=R₅=2-naphthyl, R₄=R₆=phenyl)

The same procedure as described in Preparation Example 9 was repeated but using N-phenyl-2-naphthylamine (0.85 g, 3.9 mmol) to obtain Compound (10) (0.29 g, 0.34 mmol, overall yield: 19%)

¹H NMR (200 MHz, CDCl₃): δ 6.4 (d, 4H), 6.6 (t, 2H), 6.75-6.8 (m, 8H), 7.0-7.1 (m, 6H), 7.2-7.3 (m, 6H), 7.45-7.6 (m, 10H), 7.65-7.8 (m, 6H), 7.9 (s, 2H)

MS: 864 (found), 865.10 (calculated).

### Preparation Example 11

### Preparation of Compound (11) (Chemical Formula 1: R₁=R₂=2-naphthyl, R₃=R₄=R₅=R₆=2-anthryl)

The same procedure as described in Preparation Example 1 was repeated but using di(2-anthryl)amine (2.8 g, 7.6 mmol) to obtain Compound (11) (0.29 g, 0.25 mmol, overall yield: 7%).

¹H NMR (200 MHz, CDCl₃): δ 6.75-6.8 (m, 12H), 7.25-7.3 (m, 12H), 7.45-7.6 (m, 16H), 7.65-7.8 (m, 14H), 7.9 (s, 2H)

MS/FAB: 1164 (found), 1165.46 (calculated)

### Example 1

### Manufacture of OLED by Using the Compound According to the Present Invention

An OLED having the structure employing the electroluminescent material.

First, a transparent electrode ITO thin film (15Ω/□) obtained from a glass for OLED (manufactured by Samsung-Corning) was subjected to ultrasonic washing by trichloroethylene, acetone, ethanol and distilled water, subsequently, and stored in isopronanol before use.

Then, an ITO substrate was equipped in a substrate folder of vacuum vapor deposition apparatus, and 4,4′,4″-tris(N,N-(2-naphthyl)-phenylamino)triphenylamine (2-TNATA) represented by following structural formula was placed in a cell of the vacuum vapor deposition apparatus, which was then ventilated up to 10⁻⁶ torr of vacuum in the chamber. Electric current was applied to the cell to evaporate 2-TNATA to vapor-deposit a hole injection layer having 60 nm of thickness on the ITO substrate.

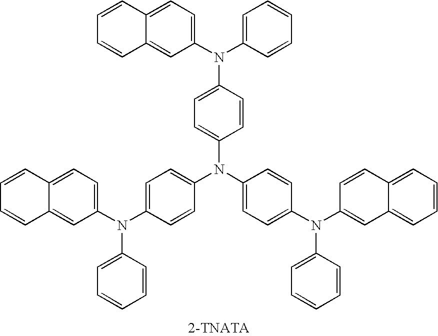

Then, to another cell of the vacuum vapor deposition apparatus, charged was N,N′-bis(α-naphthyl)-N,N′-diphenyl-4,4′-diamine (NPB), and electric current was applied to the cell to evaporate NPB to vapor-deposit a hole transport layer of 20 nm of thickness on the hole injection layer.

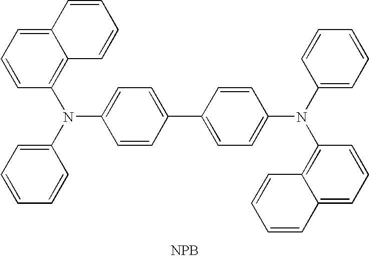

After forming the hole injection layer and hole transport layer, an electroluminescent layer was vapor-deposited thereon as follows. In one cell of the vacuum vapor deposition apparatus, charged was 7,12-di(2-naphthyl)-10-phenyl-benz(a)anthracene (DNPBA, Compound 34) represented by following structural formula, and in another cell, a compound according to the present invention (ex. Compound 4) as a dopant, and the two substances are evaporated at a different rate to dope with 2 mol % to 5 mol %, to vapor-deposit an electroluminescent layer (4) having 30 nm of thickness on said hole transport layer.

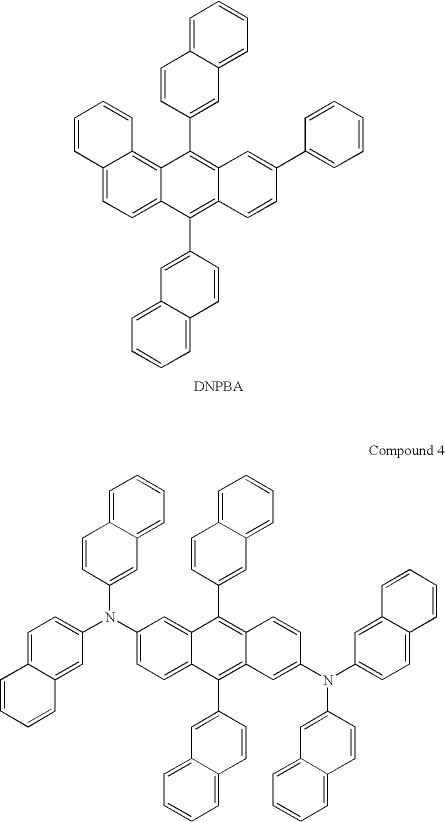

Then, Alq represented by following structural formula was vapor-deposited as an electron transport layer having 20 nm of thickness, and lithium quinolate (Liq) represented by following structural formula was vapor-deposited as an electron injecting layer having from 1 to 2 nm of thickness. Thereafter, an Al cathode was vapor-deposited with 150 nm of thickness by using another vapor deposition apparatus to manufacture an OLED.

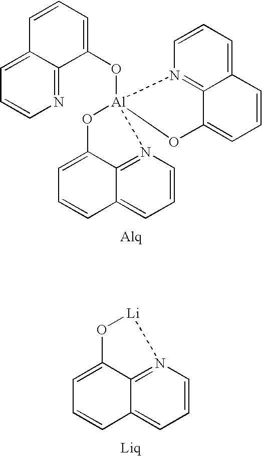

Each compound for individual material was purified by vacuum sublimation under 10⁻⁶ torr, and employed as an electroluminescent material for OLED.

### Comparative Example 1

### Preparation of an OLED Employing Conventional Electroluminescent Material

A hole injecting layer and hole transport layer were created according to the same procedure as described in Example 1, and tris(8-hydroxyquinoline)aluminum (III) (Alq) was charged as an electroluminescent host material to another cell of said vacuum vapor deposition apparatus. Coumarin 545T (C545T) represented by following structural formula was charged to still another cell. The two substances are doped by evaporating with different rates, to vapor-deposit an electroluminescent layer having 30 nm of thickness on said hole transport layer. The doping concentration at this time was preferably from 2 to 5 mol % on the basis of the amount of Alq.

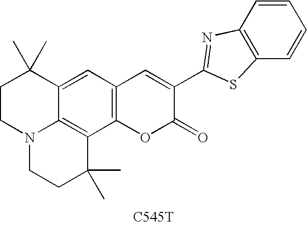

Then, an electron transport layer and an electron injecting layer were vapor-deposited according to the same procedure as described in Example 1, and an Al cathode was vapor-deposited by using another vacuum vapor deposition apparatus with a thickness of 150 nm, to manufacture an OLED.

### Comparative Example 2

### Manufacturing of an OLED by Using a Conventional Electroluminescent Material

A hole injecting layer and hole transport layer were created according to the same procedure as described in Example 1, and DNPBA was charged as an electroluminescent host material to another cell of said vacuum vapor deposition apparatus, while Compound G was charged to still another cell. The two substances are doped with from 2 to 5 mol % concentration based on DNPBA by evaporating with different rates, to vapor-deposit an electroluminescent layer having 30 nm of thickness on said hole transport layer.

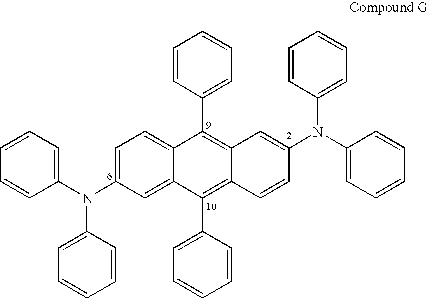

Then, an electron transport layer and an electron injecting layer were vapor-deposited according to the same procedure as described in Example 1, and an Al cathode was vapor-deposited by using another vacuum vapor deposition apparatus with a thickness of 150 nm, to manufacture an OLED.

### Example 2

### Electroluminescent Properties of OLED Manufactured

Electroluminescent efficiency of an OLED comprising the organic electroluminescent compounds prepared from Example 1 and Comparative Example 1 according to the present invention and a conventional electroluminescent compound was measured at 5,000 cd/m² and 20,000 cd/m², respectively, of which the results are shown in Table 1. Since the luminescent properties in the range of high luminance are very importance in case of green electroluminescent material, in particular, the data of luminance as high as about 20,000 cd/m² was also attached in order to reflect those properties.

TABLE 1

| | | | Doping | Efficiency | | |
|---|---|---|---|---|---|---|
| | | | Concen- | (cd/A) | | |
| | | | tration | @5,000 | @20,000 | CIE |
| No. | Host | Dopant | (mol %) | cd/m² | cd/m² | coordinate |
| 1 | 34 | 1 | 3.0 | 18.3 | 15.5 | (0.30, 0.64) |
| 2 | 34 | 2 | 3.0 | 17.7 | 15.6 | (0.29, 0.64) |
| 3 | 34 | 3 | 3.0 | 17.5 | 14.9 | (0.30, 0.64) |
| 4 | 34 | 4 | 3.0 | 19.6 | 18.1 | (0.29, 0.64) |
| 5 | 34 | 5 | 3.0 | 19.1 | 16.5 | (0.29, 0.65) |
| 6 | 34 | 6 | 3.0 | 16.5 | 14.1 | (0.29, 0.65) |
| 7 | 34 | 7 | 3.0 | 16.1 | 13.9 | (0.29, 0.65) |
| 8 | 34 | 8 | 3.0 | 19.8 | 17.2 | (0.31, 0.64) |
| 9 | 34 | 9 | 3.0 | 17.1 | 14.4 | (0.29, 0.65) |
| 10 | 34 | 10 | 3.0 | 17.4 | 14.5 | (0.29, 0.65) |
| 11 | 34 | 11 | 5.0 | 14.2 | 12.1 | (0.28, 0.66) |
| 12 | 34 | 12 | 5.0 | 13.0 | 11.1 | (0.28, 0.66) |
| 13 | 34 | 13 | 3.0 | 16.6 | 13.6 | (0.29, 0.65) |
| Comp. 1 | Alq | C545T | 2.0 | 10.3 | 9.1 | (0.29, 0.65) |
| Comp. 2 | 34 | Compound G | 3.0 | 11.0 | 8.4 | (0.26, 0.62) |

As can be seen from Table 1, 3.0% doping with Compound 34 (DNPBA) showed the highest electroluminescent efficiency. In particular, Compounds 4, 5 and 8 showed about twice of the efficiency as compared to that of conventional Alq:C545T (Comparative Example 1) or of Compound G (Comparative Example 2).

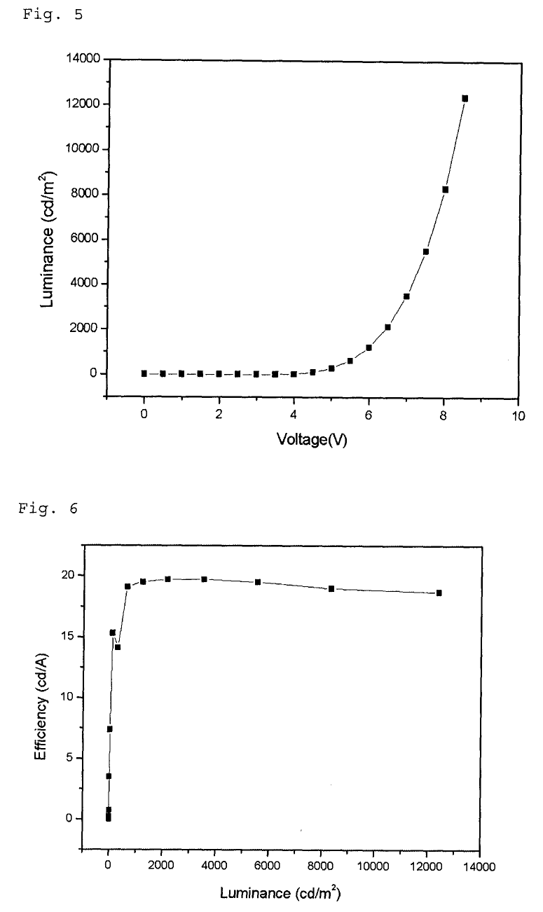

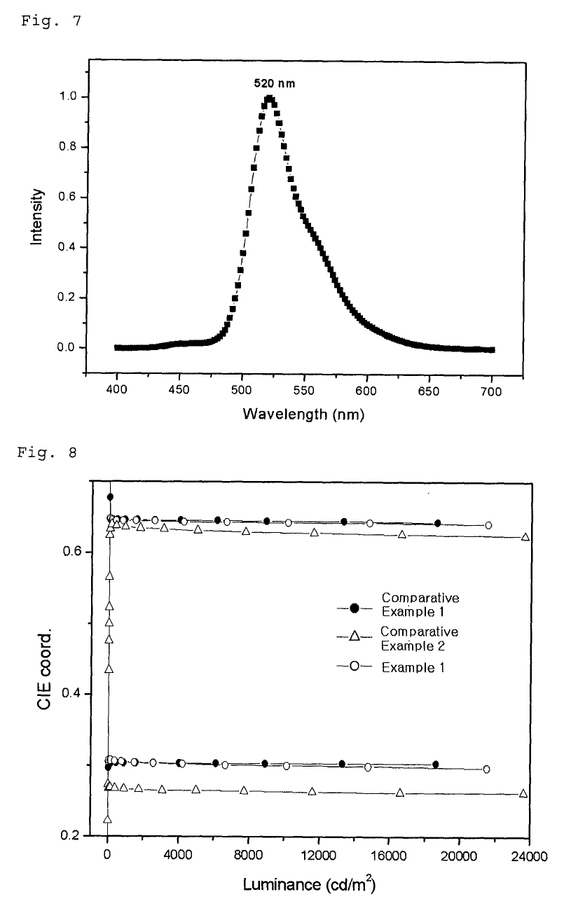

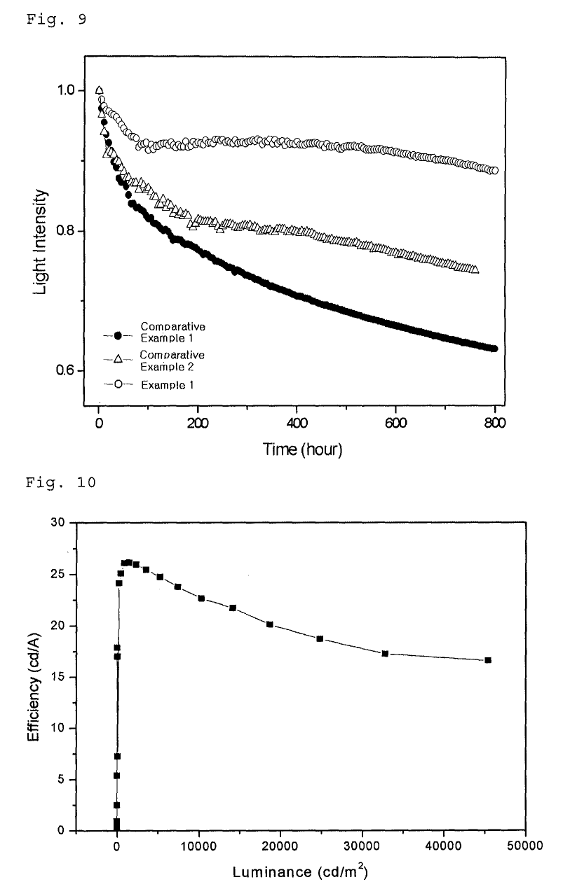

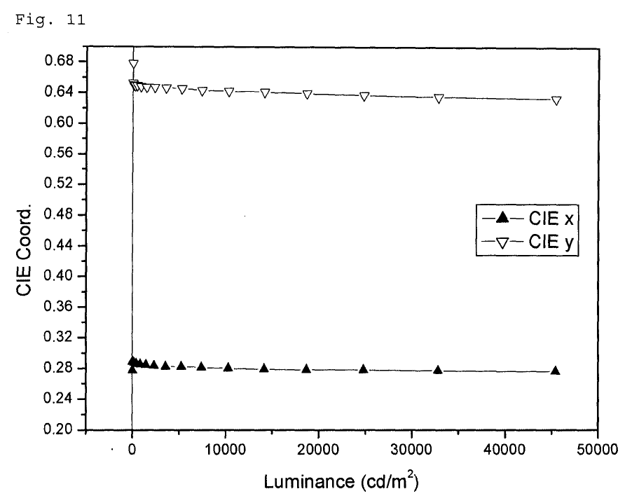

FIG. 3 is an electroluminescent efficiency curve of Alq:C545T as a conventional electroluminescent material, and FIG. 4 is an electroluminescent efficiency curve with Compound G being employed as the electroluminescent material. FIG. 5 and FIG. 6 shows luminance-voltage and electroluminescent efficiency-luminance curve, respectively. In particular, the fact that the high performance electroluminescent material according to the present invention have not more than 3 cd/A of lowering of efficiency even at a luminance as high as about 20,000 cd/m², indicates that the electroluminescent material of the present invention has excellent material properties even maintained at a high luminance.

The results of Table 1 shows that C545T also exhibits good luminescent color property, while Compound G exhibits luminescent color with short-wavelength shift, which means somewhat poor luminescent color property as compared to the materials according to the present invention. FIG. 6 is an EL spectrum of electroluminescent material according to the present invention, while FIG. 7 compares the luminescent color of Compound 4 according to the present invention and that of Comparative Example 1. They show good luminescent color properties without showing significant difference from conventional pure green electroluminescent material. They show typical green electroluminescent peak at 520 nm, and the material according to the invention shows no substantial deterioration of properties of color purity as the electroluminescent efficiency increases.

Among the properties of material according to the present invention, FIG. 9, that is a life curve at the luminance of 10,000 cd/m², shows that the life property of the material of the invention is excellent as compared to that of conventional electroluminescent material. In particular, the material according to the present invention does not have property of rapid lowering of initial luminance as was found in conventional material. Relative luminances after 800 hours of operation are 63%, 73% and 88% for C545T, Compound G and Example 1, respectively, which implies actual improvement of 2 to 5 times of life in terms of half life of luminance. This is the most valuable advantage of the material of the invention, on the contrast concept of the conventional electroluminescent material which has excellent electron conductivity.

### Example 3

### Manufacturing of OLED's Employing the Compound According to the Present Invention and Compound of Chemical Formula 3

A hole injecting layer and hole transport layer were created according to the same procedure as described in Example 1, and Compound (18) (or Compound (19) or Compound (23), or Compound (24), or Compound (25)) was charged as an electroluminescent host material to another cell of said vacuum vapor deposition apparatus. Compound (1) (or Compound (5), or Compound (13)) was charged to still another cell. The two substances are doped by evaporating with different rates, to vapor-deposit an electroluminescent layer having 30 nm of thickness on said hole transport layer. The doping concentration at this time was preferably from 2 to 5 mol % on the basis of the amount of the electroluminescent host material.

TABLE 2

| | | | Doping | Efficiency | | |
|---|---|---|---|---|---|---|
| | | | Concen- | (cd/A) | | |
| | | | tration | @5,000 | @20,000 | CIE |
| No. | Host | Dopant | (mol %) | cd/m² | cd/m² | coordinate |
| 14 | 18 | 1 | 3.0 | 25.1 | 20.1 | (0.27, 0.63) |
| 15 | 19 | 1 | 3.0 | 24.5 | 19.7 | (0.29, 0.65) |
| 16 | 23 | 1 | 3.0 | 24.7 | 19.9 | (0.28, 0.64) |
| 17 | 24 | 1 | 3.0 | 22.8 | 17.3 | (0.29, 0.65) |
| 18 | 18 | 5 | 3.0 | 24.3 | 19.5 | (0.29, 0.64) |
| 19 | 19 | 5 | 3.0 | 22.1 | 17.1 | (0.27, 0.63) |
| 20 | 23 | 5 | 3.0 | 23.9 | 18.8 | (0.29, 0.64) |
| 21 | 25 | 5 | 3.0 | 27.0 | 21.5 | (0.29, 0.64) |
| 22 | 18 | 13 | 3.0 | 25.4 | 20.2 | (0.28, 0.64) |
| 23 | 19 | 13 | 3.0 | 24.3 | 18.7 | (0.27, 0.63) |
| 24 | 23 | 13 | 3.0 | 26.3 | 20.5 | (0.28, 0.65) |
| 25 | 25 | 13 | 3.0 | 22.5 | 17.7 | (0.27, 0.63) |

From Table 2, improved properties of various electroluminescent host materials according to the present invention were confirmed.

In particular, when employing a 9,10-diarylanthracene derivative with an aromatic ring substituent at 2-position, as suggested according to the present invention, a large enhancement in terms of electroluminescent efficiency was confirmed, while no significant difference from conventional host material occurred in terms of CIE coordinates. Thus, the materials according to the present invention showed improvement at low luminance as well as high luminance, so that they can give advantageous properties in both passive- and active-type organic light emitting diode. Actually, the properties as described above are advantageous in terms of electric power consumption as compared to the case employing conventional 9,10-diarylanthracene as the electroluminescent host material, to afford the present invention of many chances to be practiced.

### INDUSTRIAL APPLICATION

The organic electroluminescent compounds according to the present invention have good electroluminescent efficiency and excellent life properties, thereby providing OLED having very long lifetime of operation.

1. An organic electroluminescent compound represented by Chemical Formula 1 or 2: 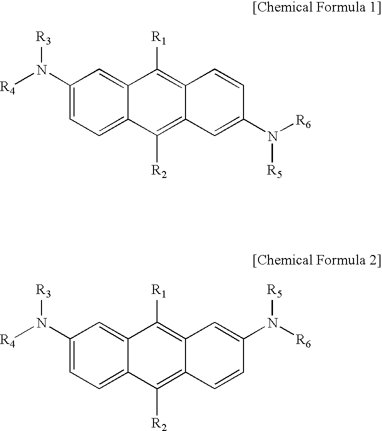
wherein R₁ and R₂ independently represent a fused multi-cyclic aromatic ring having two or more aromatic rings fused therein, and R₃ to R₆ independently represent an aromatic ring, and each aromatic ring of R₁ to R₆ may be further substituted by a C1-C20 alkyl group, a C1-C20 alkoxy group, a halogen atom or a C5-C7 cycloalkyl group.

2. An organic electroluminescent compound according to claim 1, wherein each of R₁ and R₂ of Chemical Formula 1 or 2 is independently selected from naphthyl, anthryl, fluoranthenyl, pyrenyl, fluorenyl, biphenyl and perilenyl group; and each of R₃ to R₆ is independently selected from phenyl, naphthyl, anthryl, phenanthryl, fluorenyl, fluoranthenyl, pyrenyl, perilenyl, naphthacenyl and biphenyl group.

3. An organic electroluminescent compound according to claim 2, wherein each of R₁ and R₂ of Chemical Formula 1 or 2 is independently selected from 2-naphthyl, 2-anthryl, 2-fluoranthenyl, 1-pyrenyl, 2-fluorenyl, 4-biphenyl and 3-perilenyl group.

4. An organic electroluminescent compound according to claim 3, wherein each aromatic ring of R₁ to R₆ has additional substituent of methyl, t-butyl or methoxy group.

5. An organic electroluminescent compound according to claim 4, which is represented by one of the following structural formulas:

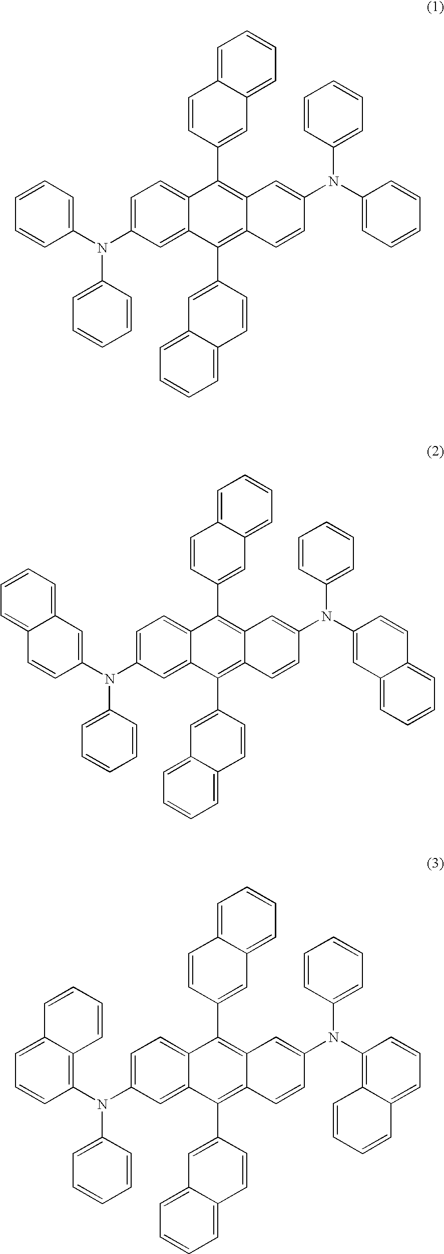

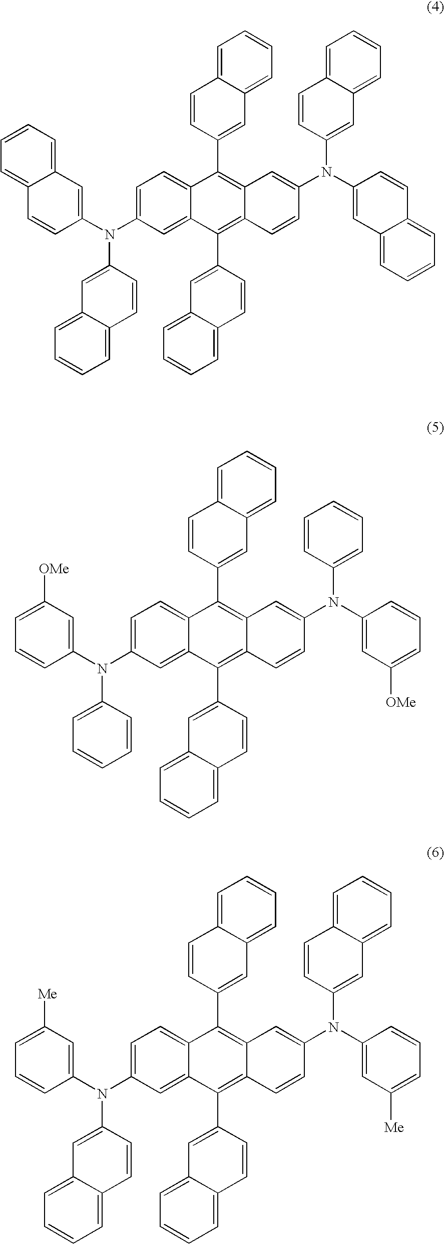

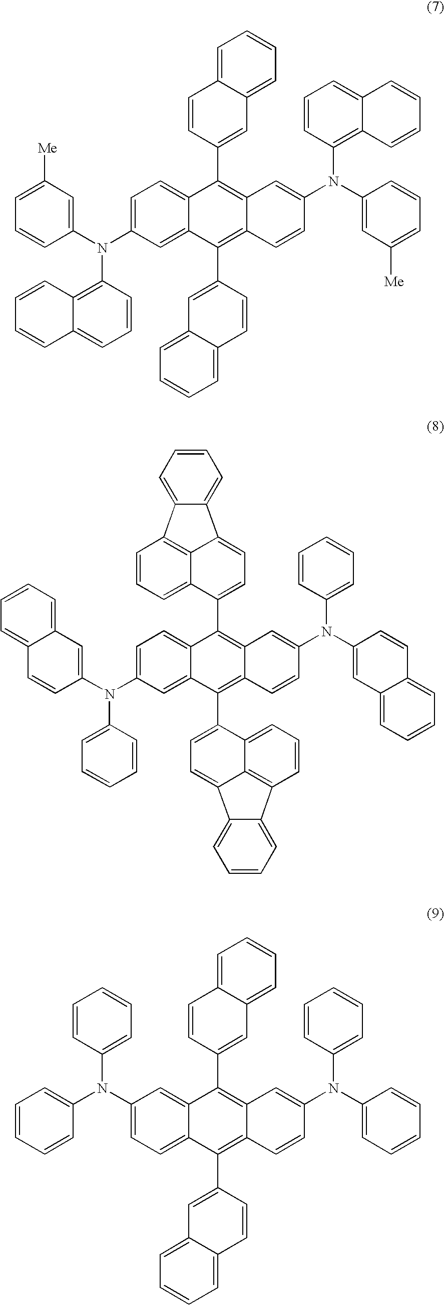

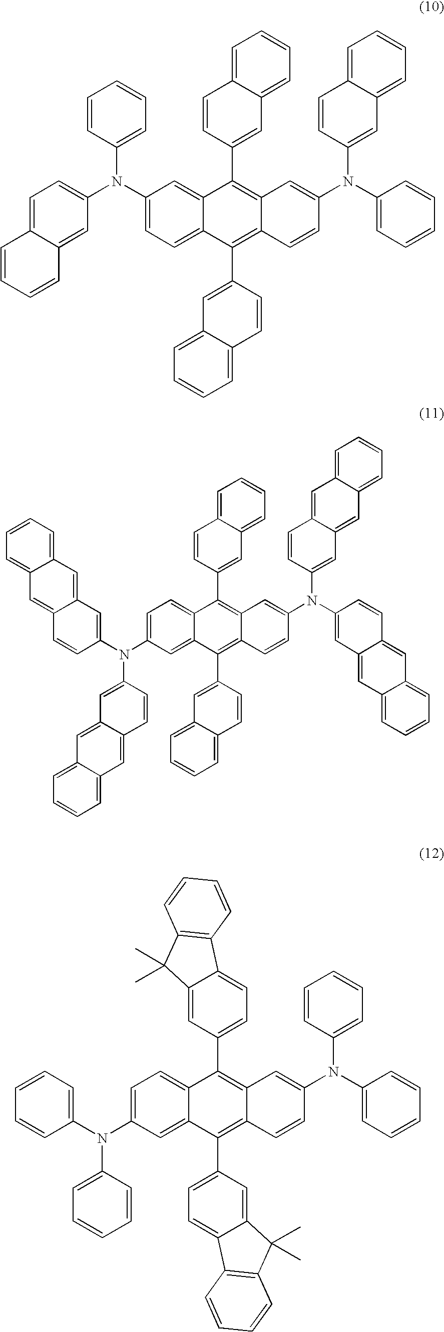

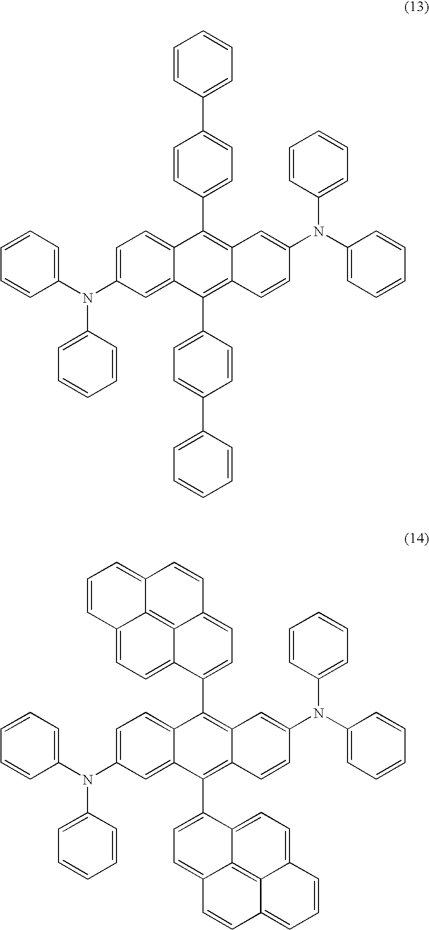

6. An organic light emitting diode (OLED) comprising a first electrode, at least one organic layer and a second electrode in a subsequently laminated form, wherein at least one layer of said organic layers comprise(s) the organic electroluminescent compound according to claim 1.

7. An organic light emitting diode (OLED) comprising an anode; a cathode; and a luminescent region interposed between said anode and said cathode, wherein said luminescent region comprises at least one organic electroluminescent compound according to claim 1; and at least one compound selected from anthracene derivatives, benz[a]anthracene derivatives and naphthacene derivatives.

8. An organic light emitting diode (OLED) according to claim 7, wherein the anthracene derivative or benz[a]anthracene derivative is a compound represented by Chemical Formula 3 or 4: 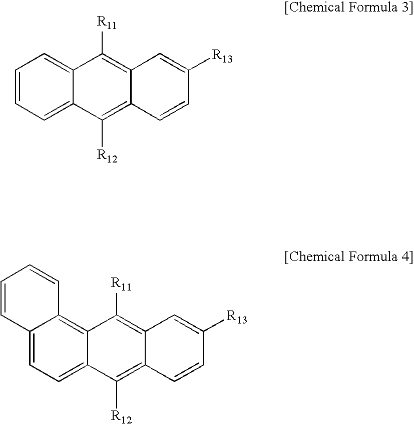
wherein, each of R₁₁ and R₁₂ independently represents a C6-C20 aromatic ring or a fused multi-cyclic aromatic ring, R₁₃ represents a hydrogen, a C1-C20 alkyl group, a C1-C20 alkoxy group, a halogen atom, a C5-C7 cycloalkyl group, or a C6-C20 aromatic ring or a fused multi-cyclic aromatic ring, and each aromatic ring of R₁₁ to R₁₃ may have further substituent(s) of a C1-C20 alkyl group, a C1-C20 alkoxy group, a halogen atom or a C5-C7 cycloalkyl group.

9. An organic light emitting diode (OLED) according to claim 8, wherein each of R₁₁ to R₁₃ of Chemical Formula 3 or 4 is independently selected from phenyl, 2-naphthyl, 2-anthryl, 2-fluoranthenyl, 1-pyrenyl, 2-fluorenyl, 4-biphenyl and 3-perilenyl group.

10. An organic light emitting diode (OLED) according to claim 9, wherein the anthracene derivative is a compound represented by one of the following formulas: 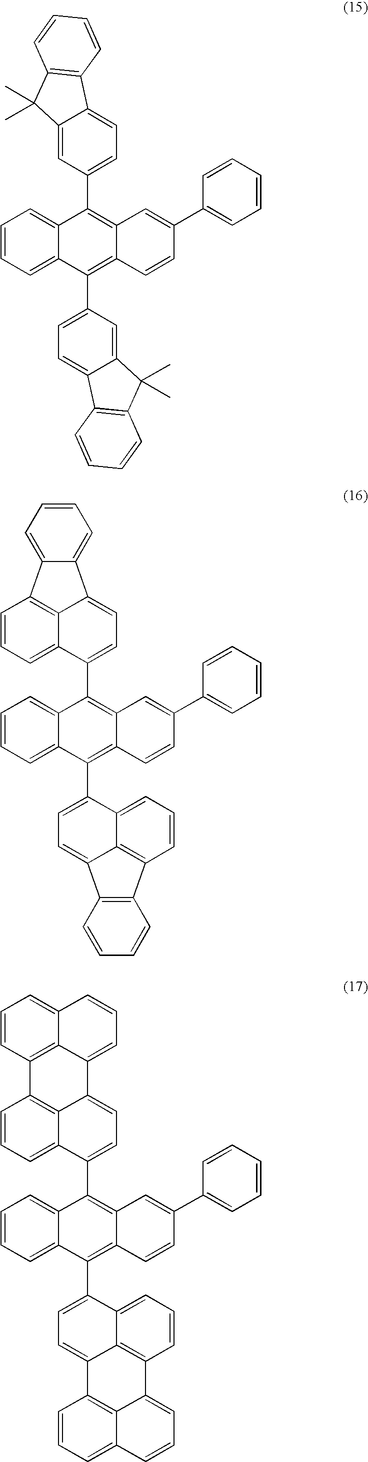 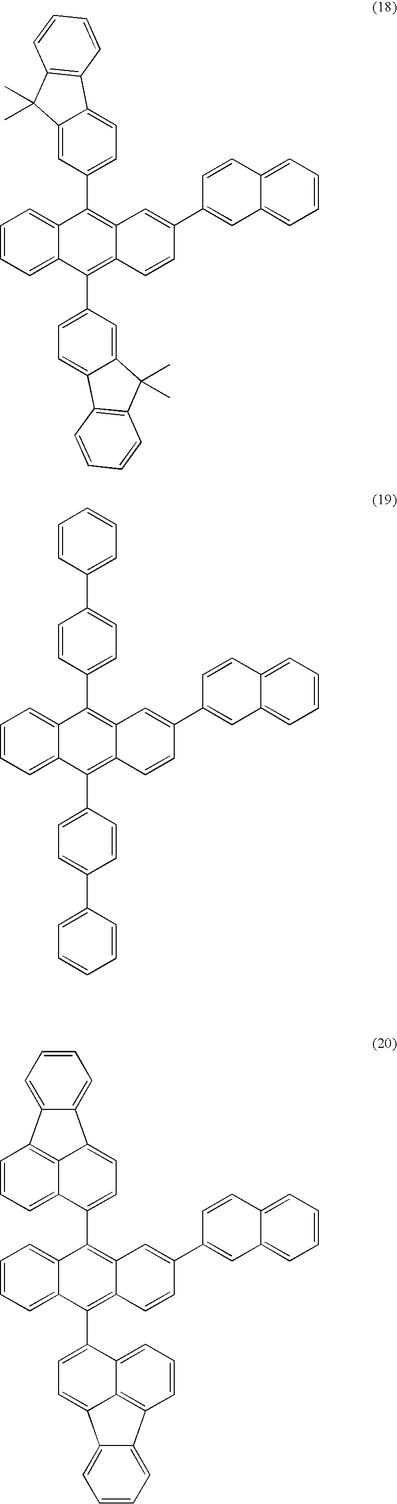 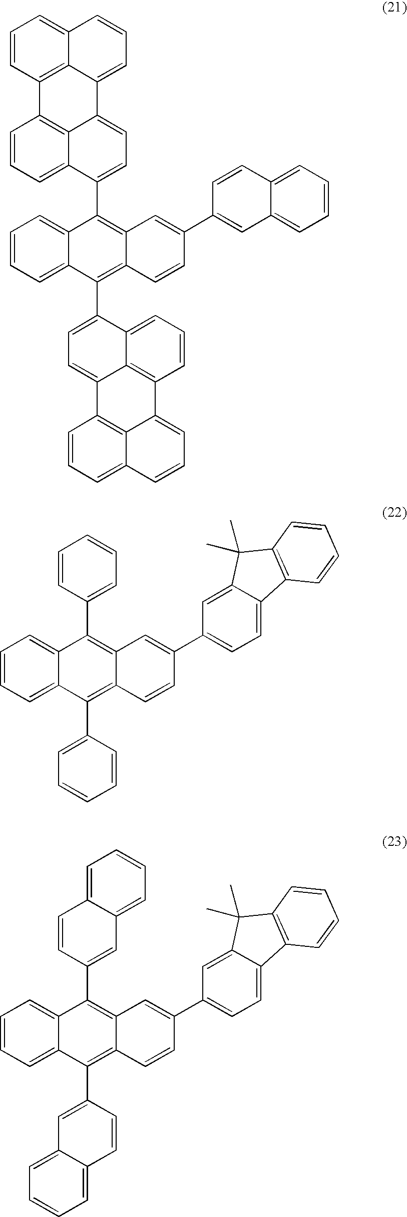 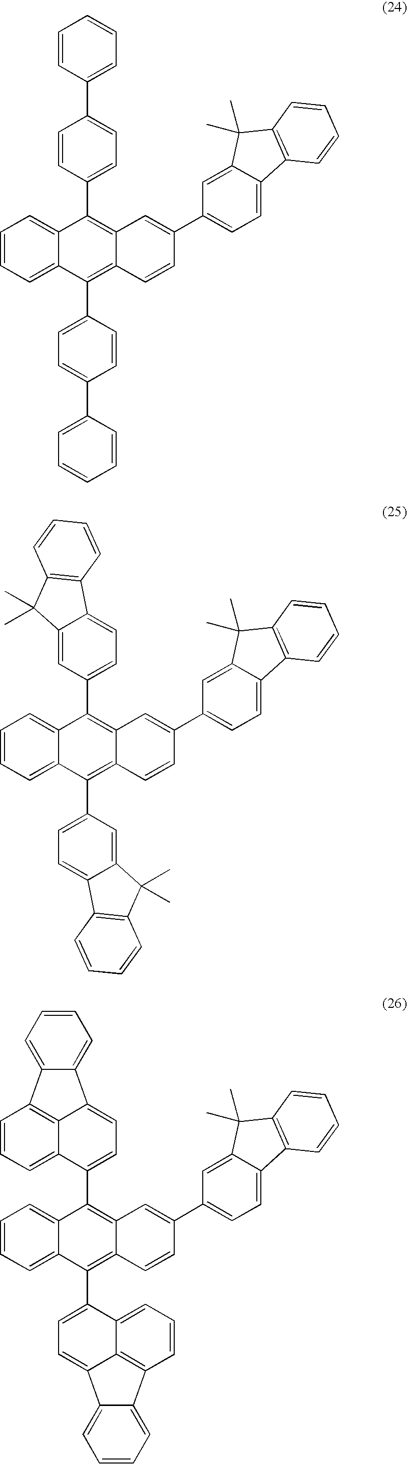 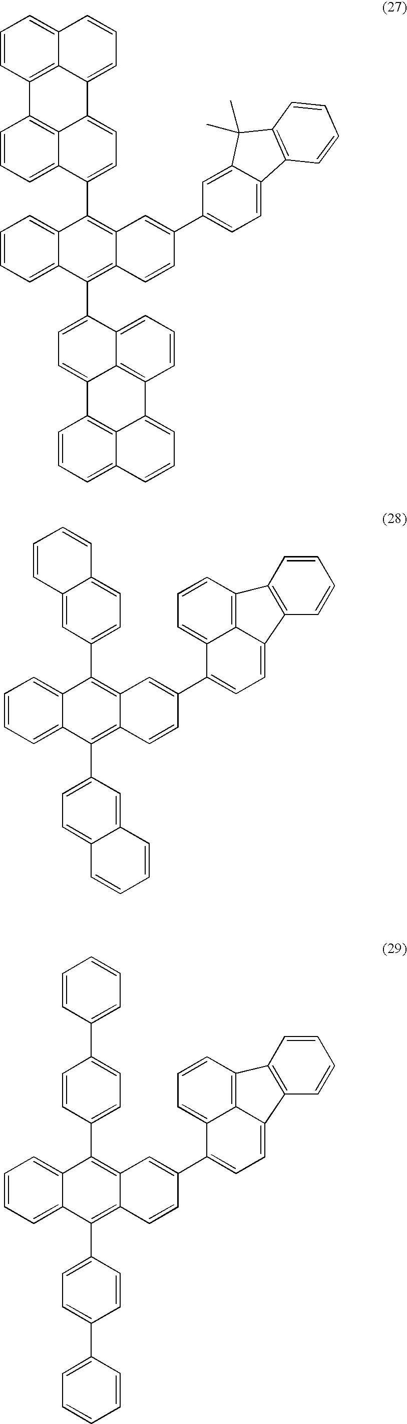 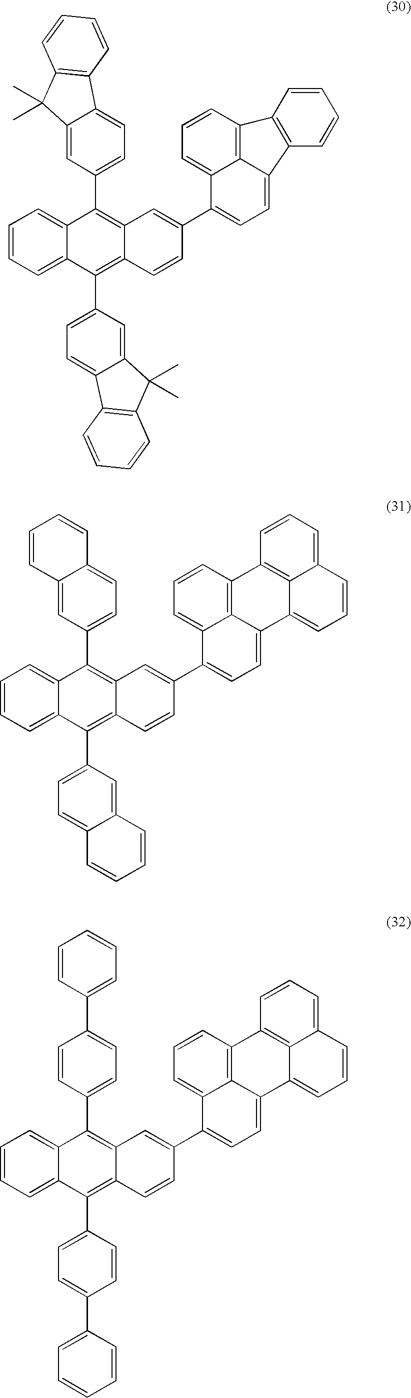 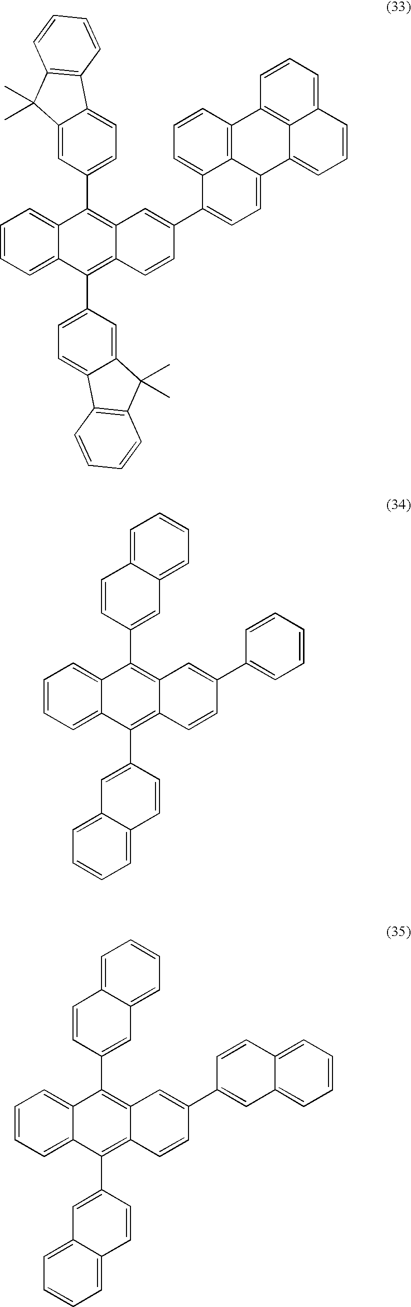 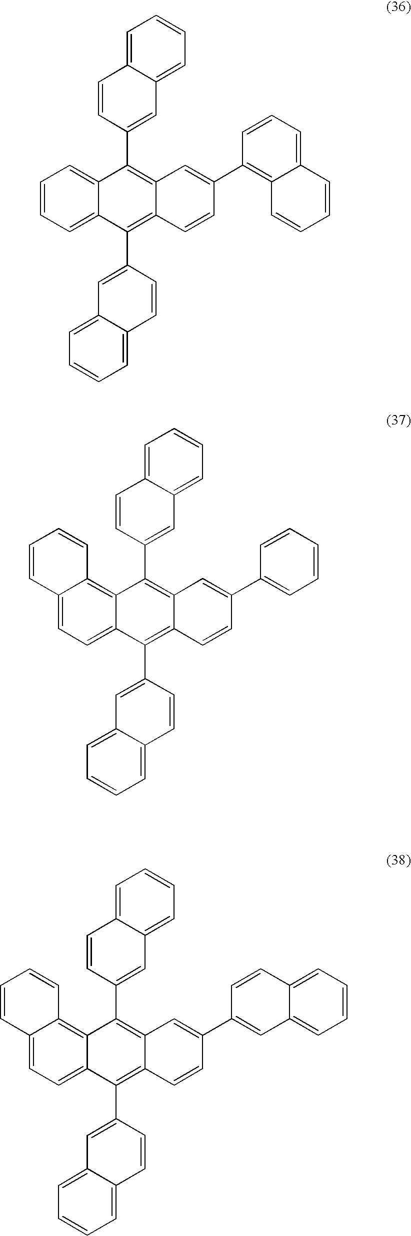 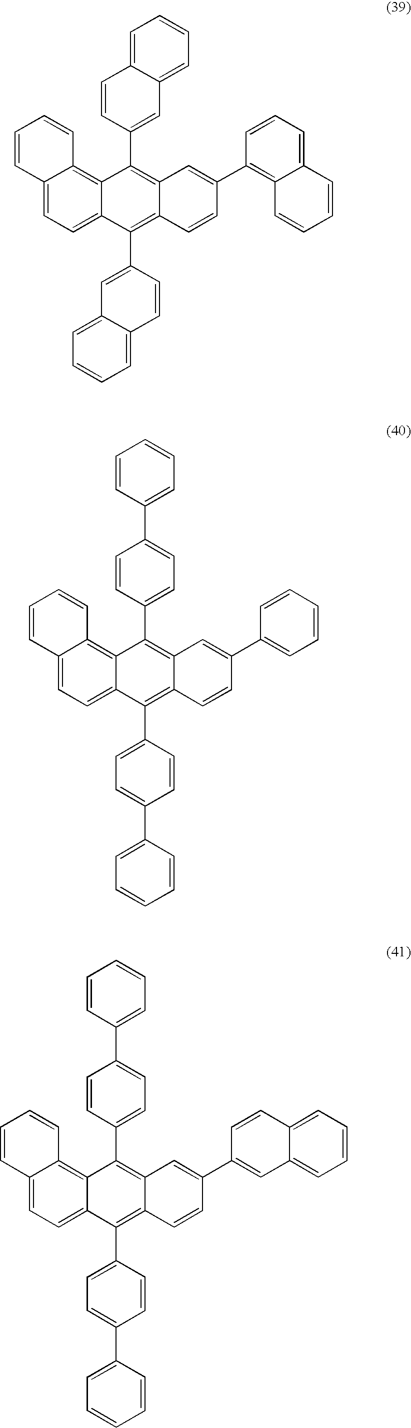 [chemical structure]
